(19) United States
(12) Patent Application Publication
(10) Pub. No.: US 2021/0393677 A1
(43) Pub. Date: Dec. 23, 2021

(54) COMPOUNDS FOR MODULATION AND AS FUNCTIONAL REPLACEMENT OF ALPHAKETOGLUTARIC ACID (2OG)-DEPENDENT OXYGENASES

(71) Applicant: Thomas HOMANN, Schildow (DE)

(72) Inventor: Thomas HOMANN, Schildow (DE)

(21) Appl. No.: 17/279,331

(22) PCT Filed: Aug. 12, 2019
(86) PCT No.: PCT/EP2019/071620

§ 371 Date: Mar. 24, 2021

(30) Foreign Application Priority Data
Aug. 10, 2018 (LU) LU100900

(51) Int. Cl. A61K 33/26; A61K 45/06; C07F 15/02; A61P 35/00

(52) U.S. Cl. CPC A61K 33/26; A61P 35/00; C07F 15/025; A61K 45/06

(57) ABSTRACT

The present invention relates to an alternative co-substrate of ketoglutaric acid-dependent dioxygenases for functional production and control of same, with the aim of achieving therapeutic effects against cancer, neurodegenerative diseases and age related diseases. Epigenetically induced diseases caused by dysregulation and in particular also by metabolic dysfunction in the citric acid cycle are likewise targeted by this therapy.

### FIELD OF THE INVENTION

The invention relates to compounds as functional equivalents of the co-substrate α-ketoglutaric acid (synonymously also referred to as 2-oxoglutarate or 2-OG) in 2-OG-dependent oxygenases (DIOs), in particular for the functional production thereof in presence of competitive inhibitors of the DIOs, such as, for example, 2-hydroxoglutaric acid (2-HG).

### BACKGROUND OF THE INVENTION

α-Ketoglutarate-(2-oxoglutarate, 2-OG)-dependent dioxygenases (DIOs, also referred to as 2-OG-oxygenases) are oxygen-dependent enzymes that use α-ketoglutaric acid (2-oxoglutarate 2-OG, 2-KG) as co-substrates and non-heme Fe(II) as co-factor. They catalyze numerous oxidation reactions. They include (not exclusively) hydroxylation reactions, demethylations, ring expansions, cyclizations, and introduction of double bonds. [1] [2, 3].

2-OG-dependent dioxygenases are involved in many biological processes and functions [4, 5]. In microorganisms such as bacteria, 2-OG-dependent dioxygenases are involved in basic functions for biosynthesis [6-8]. In plants, 2-OG-dependent dioxygenases are involved in many different reactions of the plant metabolism [9]. Here are included (but not exclusively) the flavonoid biosynthesis and ethylene biosyntheses [10]. In mammals and humans, 2-OG-dependent dioxygenases have functional roles in biosyntheses (e.g., collagen biosynthesis [11] and L-carnitine biosynthesis [12]), post-translational modifications (e.g., protein hydroxylation [13]), epigenetic regulations (e.g., histone and DNA demethylation [14]) and sensors of the energy metabolism [15].

2-OG-dependent dioxygenases catalyze oxidation reactions by incorporating a single oxygen atom into their substrates. This is always accompanied by the oxidation of the co-substrate 2-OG to succinate and carbon dioxide [16].

The catalytic activity of many 2-OG-dependent dioxygenases is to be dependent on reduction agents that are capable to keep the co-factor iron in its divalent form or to reduce it to this oxidation state [1] [17-20]. This mechanism is also discussed for vitamin C (ascorbate). However, not all of the functions can be explained thereby [21].

2-OG-dependent dioxygenases are characterized by a common catalytic mechanism. In the first step, the binding of 2-OG and substrate into the active binding site occurs [22-24]. 2-OG is directly coordinated with iron (II) (Ni II; Mn II) in the activity center, whereas the substrate binds in immediate proximity, but not directly coordinating, to the metal. The second step is the binding of molecular oxygen that assumes a third site at the Fe(II) (NiII; MnII) center. This enables an oxidative decarboxylation reaction under the formation of succinate, carbon dioxide, and a reactive metal(IV)-oxo-intermediate that subsequently oxidizes the substrate [25-31]. The replacement and the role of iron were the focus of investigations [59].

An alternative mechanism was discussed in 2004 for a bacterial 2-OG-dependent dioxygenase, deacetoxycephalosporin-C synthase (DAOCS). The proposed “ping-pong” mechanism differs from the consensus mechanism (supra) in that 2-OG and oxygen are first bound to the Fe(II) center in the enzyme active site in absence of the substrate. The decoupled oxidation of 2-OG then occurs for the production of a reactive Fe(IV)-oxo species, followed by the release of succinate and carbon dioxide and the binding of the substrate, which is oxidized. Later studies in 2014 showed, however, that the DAOCS also follows, with high likelihood, the general consensus mechanism of the 2-OG-dependent oxygenases [32].

All 2-OG-dependent dioxygenases contain a conserved double-strand β-helix (DSBH) that forms, with two β-sheets, a cleft [33, 34]. The active site contains a highly conserved 2-His-1-carboxylate (HXD/E . . . H) amino acid residue triad motif, in which the catalytically essential metal of two histidine residues and an aspartic acid or glutamic acid residue is fixed [35]. However, they differ in the amino acid arrangement in the active center.

Results from X-ray crystallography, molecular dynamics (MD) calculations, and NMR spectroscopy show that some 2-OG-dependent dioxygenases bind their substrate via an induced adaptation mechanism. For example, protein structure alterations were observed in the substrate-binding for the human prolyl hydroxylase isoform 2 (PHD2) [36, 37], [38] a 2-OG-dependent dioxygenase that is involved in the oxygen homeostasis [39], and the isopenicillin N synthase (IPNS), a microbial 2-OG-dependent dioxygenase [40].

In many diseases, the activity of DIOs is modified, frequently also reduced. In view of the important biological role that the 2-OG-dependent dioxygenase plays, they are important targets for the therapy of diseases.

The aspect of using small molecules as a functional replacements of 2-OG as co-substrates was not known up to now in this function and is a completely new pharmacological approach for affecting DIOs for the treatment of diseases. Thus, symptoms that are caused by a shortage of 2-OG or by competitive inhibition by oncometabolites (e.g., 2-HG) in the enzyme, become treatable.

α-Ketoglutaric acid (2-OG)-dependent oxygenases (DIOs) catalyze a remarkably wide range of oxidative reactions. For humans and animals, these are hydroxylations and N-demethylations that take place via hydroxylation reactions; for plants and microbes, they catalyze reactions such as ring formations, rearrangements, desaturations, and halogenations.

In their biological function, the catalytic flexibility of the DIOs is reflected. After the role of DIOs in collagen biosynthesis has been identified, it could be shown that they also play a role in the development of plants and animals, the transcription regulation, the modification/repair of nucleic acids (DNA, RNA), the fatty acid metabolism, the formation and stabilization of stem cells (PS iPS), and the biosynthesis of secondary metabolites, including medically important antibiotics.

### OBJECT OF THE INVENTION

The present invention provides compounds for the takeover of functions of the co-substrate 2-OG in 2-OG-dependent oxygenases and the use of the same in the treatment of diseases that are associated with the 2-OG-dependent oxygenases (DIOs).

### SUMMARY OF THE INVENTION

The present invention provides compounds, wherein the compounds are selected from the compounds according to Formula (I) or Formula (II)

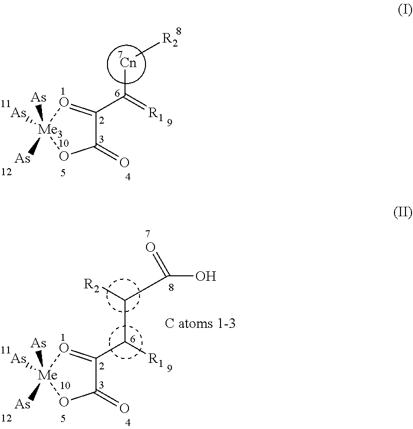

wherein
As represent the amino acids from the binding pocket of the enzyme;
Me is a metal from the catalytic center;
R₁ and R₂ are oxygen (hydroxyl) or carboxyl groups, halogens, in particular, fluorine, chlorine, or iodine, a mono- or poly-halogenated methyl group, in particular, CH₂F up to CF₃; and
Cn represents a C atom, a heteroatom, or the bridge to a heterocycle,
   that are characterized by that they can assume a reactive distance to the catalytic center and thereby can form, for the necessary function as a co-substrate, the corresponding transition states (TS).

Further, it is provided, in the compound, that R₁ is hydrogen or a CH₂R₃ group, wherein R₃ is hydrogen or oxygen (hydroxyl, carbonyl) or a shorter C-chain (C₁ to C₄).

In another aspect, the compound may be part of a ring system, wherein the ring size is between 3 and 5 atoms with at least one heteroatom.

In another embodiment of the compound, C₇ may consist of a carbon chain with up to 5 atoms and may contain double bonds.

The compound may further comprise R₂ as a carboxylic acid.

For a compound according to Formula (II), R₂ may represent a hydrogen atom, a methyl group, an alkyl group with up to 6 C atoms that may be branched saturated or unsaturated or may themselves also contain a heteroatom.

Further, for a compound according to Formula (II), it is provided that between R₁ and R₂, a bridge-forming cyclic structure is arranged, comprising single or double bonds and heteroatoms.

According to the invention, a mixture of the compounds according to Formula (I) and Formula (II) is also provided.

In another aspect, pharmaceutically acceptable salts and tautomers of the respective compound are used alone or in combination in predefined mixing ratios.

Another object of the present invention is the use of the previously described compounds as drugs.

Further, an object of the invention is the use of a compound as described above as a functional co-substrate with other active ingredients in a drug, wherein the other active ingredients may be selected from, however, are not limited to, the group comprising chemotherapeutics, cytostatics (alkylants, antimetabolites, topoisomerase inhibitors, mitose inhibitors, antibiotics, antibodies, kinase inhibitors, proteasome inhibitors and supportive medicinal substances of the tumor therapy such as interferons, cytokines, tumor necrosis factor, and IDH inhibitors).

The invention further comprises the use of a compound, as described above, as a drug for the prevention, treatment, or follow-up of cancer diseases, of neurodegenerative diseases, and of congenital or acquired metabolic disorders.

Another subject matter of the present invention is the use of a compound according to Formula (I) or Formula (II) that shows the TS in the enzyme, for the preparation of a drug for the prevention, treatment, or follow-up of cancer diseases, of neurodegenerative diseases and of congenital or acquired metabolic disorders.

Finally, the present invention also comprises a drug comprising a compound according to Formula (I) or Formula (II).

### SHORT DESCRIPTION OF THE FIGURES

The present invention is described and illustrated with reference to figures and embodiments. For the responsible person skilled in the art, it is apparent that the invention is not limited to the contents of the figures and embodiments. There are:

FIG. 1 binding conditions that are necessary for the function as a co-substrate in the enzyme;

FIG. 2 TET2 enzyme, binding of the TET inhibitor NGA (N-oxalylglycine);

FIG. 3, 4 binding conditions of 2-OG in the TET enzyme;

FIG. 5A, 5B function loss of TET enzyme by competition of 2-HG and 2-OG;

FIG. 6 functional replacement of the co-substrate 2-OG by DKA;

FIG. 7 known therapies and the new pharmacological concept according to the present invention;

FIG. 8 toxicity of DKA;

FIG. 9 toxicity of 2-OG;

FIG. 10 combination of IDH inhibitors with co-substrates according to the present invention;

FIG. 11 correlation 2-hydroxyglutarate level with 5-hmdC levels in IDH1-MT HCT116;

FIG. 12 recovery function TET enzyme by DKA in presence of the competitive inhibitor 2-HG;

FIG. 13-18 selected chemical compounds that may serve as functional replacements of the co-substrate 2-OG at TET and HIF;

FIG. 19-27 selected chemical compounds as functional replacements for the HF enzyme;

FIG. 28 recovery of the functionality by means of DKA;

FIG. 29 MTT cytotoxicity assays;

FIG. 30, 31 apoptosis assays with 2,3-diketogulonic acid;

FIG. 32, 33 hmdC measurements with 2,3-diketogulonic acid;

FIG. 34 western blot with HCT116 cells.

### DETAILED DESCRIPTION OF THE INVENTION

The present invention relates to an alternative co-substrate at ketoglutaric acid-dependent dioxygenases ([1] [6, 7] Tab. 1-2; Tab. 4) for the functional production and regulation thereof with the aim of achieving therapeutic effects against cancer, neurodegenerative, and age-related diseases. Epigenetically induced diseases caused by dysregulation and in particular also by metabolic dysfunctions in the citric acid cycle are likewise in the focus of this therapy.

The term co-substrate refers, in the present description of the invention, to low-molecular chemical compounds that are needed for an enzymatic reaction to enable the reaction of the actual substrate. Thus, co-substrates serve as a kind of “auxiliary molecules” that are reacted together with the substrate, but do not have any own catalytic effect.

Generally, in therapies of diseases, active ingredient receptors are affected by inhibition of the target structures (competitively, non-competitively, allosterically, covalently). Further, in the DIOs, this aim is pursued by, e.g., inhibition of the IDHs or α-HIF with partially inconsistent results and toxic events [8-13]. The present invention relates to a novel kind of affecting enzymes for disease therapies, the functional replacement of the native co-substrate by one of our substances.

Regulation of the DIOs by corresponding co-substrate replacement (modulators) can optimize this, and this goes so far that targeted influence on epigenetic regulations, recovery, and regulation of the cell metabolism and the further genetic regulations associated therewith can be taken.

Further treatable are DIO-dependent orphan diseases such as 2-hydroxy-glutaric aciduria.

An after-treatment of conventionally treated tumors is another option of the application.

The present invention provides compounds for the replacement and thus for the modulation or regulation of α-ketoglutaric acid (2-OG)-dependent oxygenases. The compounds according to the invention can be used in the treatment of diseases that are dependent on the function (activity) of the α-ketoglutaric acid at dioxygenases (DIOs). These diseases include in particular cancer, Alzheimer's disease, Morbus Parkinson, age-related diseases.

A variation of the compounds provided by the invention, which are also referred to, in the context of the description of the invention, as co-substrates, enables a regulation and adaptation to the respective target structures in case of an indication.

The invention provides the following compounds of Formula (I) and Formula (II): The compound according to Formula (I) may be structured as follows:

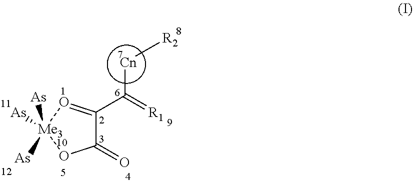

wherein
As represent the amino acids from the binding pocket of the enzyme, and Me represents a metal from the catalytic center;
R₁ and R₂ may be oxygen (hydroxyl) or carboxyl groups, halogens, in particular fluorine, chlorine, or iodine, a mono- or poly-halogenated methyl group, in particular, CH₂F up to CF₃;
R₁ may be singly bound hydrogen or a CH₂R₃ group, wherein R₃ is hydrogen or oxygen (hydroxyl, carbonyl) or a shorter C-chain (C₁ to C₄).

An example of a compound according to the invention is shown in the following:

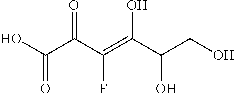 [SMILES: O=C(O)C(=O)/C(F)=C(\O)C(O)CO]

The compound according to Formula (I) may be part of a ring system, wherein the ring size contains 3 to 5 atoms with one or more heteroatoms. C₇ may consist of a carbon chain with up to 5 atoms and may contain double bonds as shown in the following example:

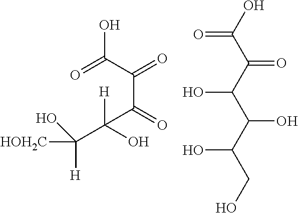

### 2,3-diketogulonic acid DKA 2-ketogulonic acid 2-OKG (2-KG)

This chain may also be included in a ring system as follows:

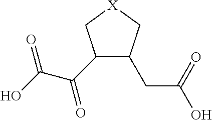

Cn in Formula (I) represents at least one C atom, a heteroatom, or the bridge to a heterocycle containing one or more heteroatoms.

R₂ represents a carboxylic acid, Cn may represent one, two or more C atoms as in 2-oxoadipic acid:

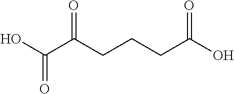 [SMILES: O=C(O)CCCC(=O)C(=O)O]

or oxobutanedioate

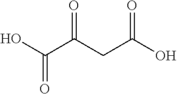

R₂ and Cn may represent an unsaturated or saturated ring with or without heteroatoms.

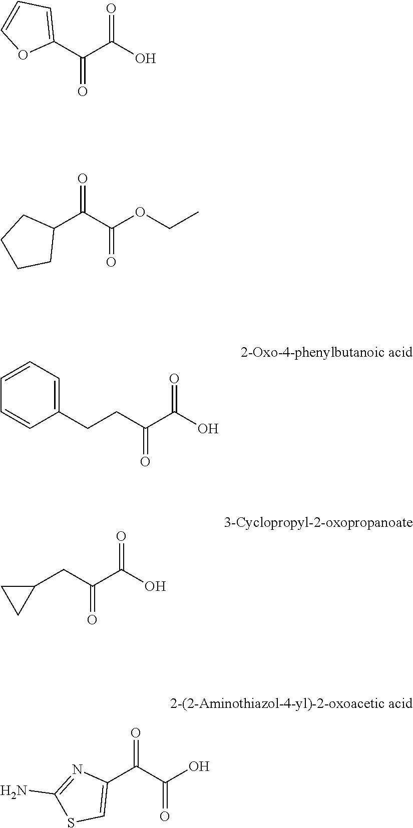

The compound according to Formula (II) may be structured as follows:

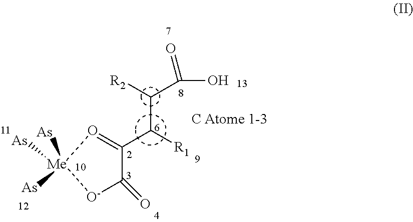

wherein
As represent the amino acids from the binding pocket of the enzyme, and Me represents a metal from the catalytic center;
R₁ and R₂ may be oxygen (hydroxyl) or carboxyl groups, halogens, in particular fluorine, chlorine, or iodine, a mono- or poly-halogenated methyl group, in particular, CH₂F up to CF₃;

R₁ may be singly bound hydrogen or a CH₂R₃ group, wherein R₃ may be hydrogen or oxygen (hydroxyl, carbonyl) or a shorter C-chain (C₁ to C₄).

The compound according to Formula (II) may be part of a ring system, wherein the ring size contains 3 to 5 atoms with one or more heteroatoms. C₇ may consist of a carbon chain with up to 5 atoms and may contain double bonds, as exemplarily shown in the following:

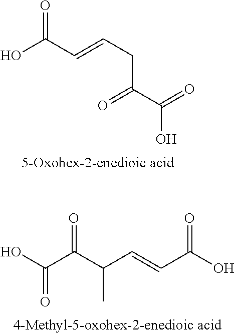

This chain may also be included in a ring system:

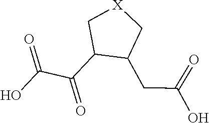

Cn in Formula (II) represents a C atom or a heteroatom.

R2 represents a carboxylic acid, Cn may represent one, two or more C atoms, such as for example in 2-oxoadipic acid (supra), or oxobutanedioate (supra).

Cn may be part of or a bridge to a heterocycle that contains one or more heteroatoms, such as, e.g., in:

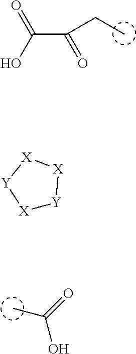

Ring Size R 4-8

In Formula (II), R₁ may be a hydrogen atom, a methyl group, or an alkyl group with up to 5 C atoms that may be branched saturated or unsaturated or may also contain heteroatoms.

R₂ may also be a hydrogen atom, a methyl group, an alkyl group with up to 6 C atoms that may be branched saturated or unsaturated or may also contain a heteroatom, as outlined in the following examples.

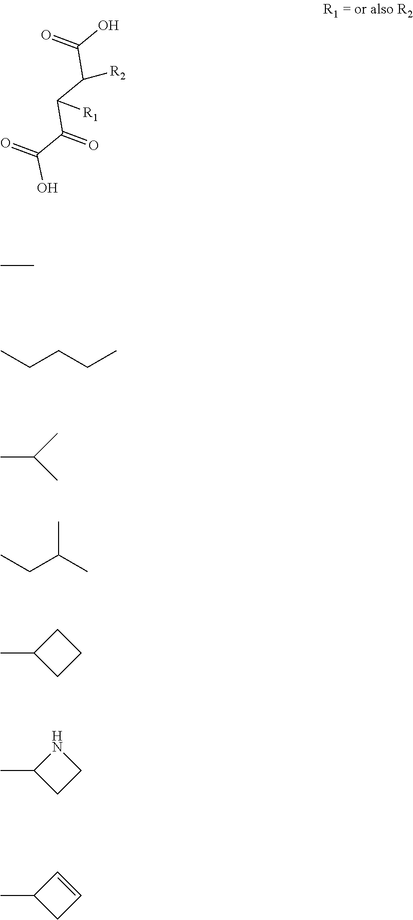

Between R₁ and R₂ in Formula (II), a bridge-forming cyclic structure may be arranged that may be unsaturated or saturated or contain heteroatoms. The formed cycle may be a 3-ring, a 4-ring, a 5-ring or also a 6-ring. The ring may contain one or more double bonds, likewise in addition thereto or alone one or more heteroatoms of the same kind or also mixed, as outlined in the following examples.

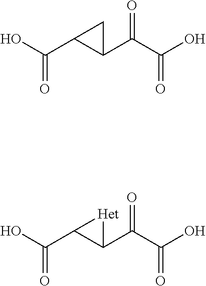

One double bond, also in connection with heteroatoms

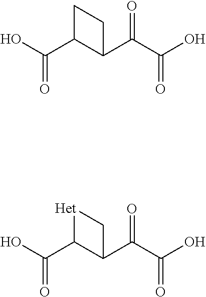

One or more double bonds, also in connection with heteroatoms

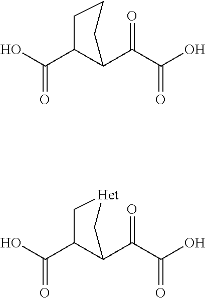

One or more double bonds, also in connection with heteroatoms

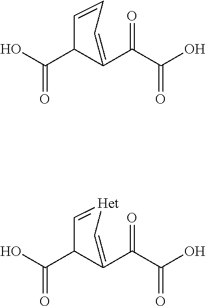

One or more double bonds, also in connection with heteroatoms

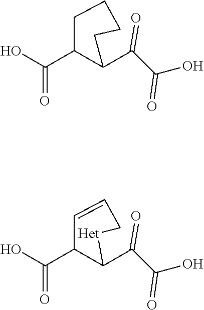

One or more double bonds, also in connection with heteroatoms.

The chain of the C atoms can be extended, and the substituents can be employed as shown in the following examples.

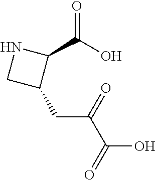 [SMILES: O=C(O)C(=O)C[C@@H]1CN[C@H]1C(=O)O]

The compounds may be employed as a substance, as a salt and in a buffered form. The carboxylic acids may also be employed in an esterified form, with the esterification also with higher-chain alcohols (up to C₁₂ atoms) being possible.

Processing takes place on a pharmaceutical-technical level. Preparations such as, e.g., creams, ointments, gels, nanoformulations, infüsion solutions, tablets, capsules are possible.

As a classic prodrug, vitamin C can be envisioned, which can be metabolized in the body to compounds according to Formula (I) or according to Formula (II) and to the structure according to Formula (II) shown in the following. Esterification of the carboxyl group also leads to prodrugs with improved resorption and cell absorption. Further options for a prodrug design are listed in [48].

The combination of prodrug and replacement co-substrate (modulator) is possible. Administration may be performed orally, locally, or by infusion.

The mixture or combined administration of a classic tumor therapeutic and a modulator for the efficiency increase of the therapy is possible and represents an optimization of the therapy.

For the claimed structural elements, numerous known structures (CAS) are available that, depending on the DIOs, can be provided as modulators, and the DIOs can be affected thereby.

Likewise, structures are available for the selection that work as prodrugs. The simplest example is vitamin C and the derivatives thereof, such as 2-O-α-D-glucopyranosyl-1-ascorbic acid that can be metabolized to 2,3-diketogulonic acid (DKA) and also to 3,4,5-trihydroxy-2-oxopentanoic acid (III; 2-KGL)

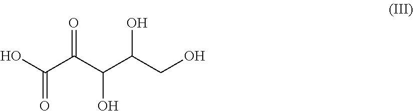 [SMILES: O=C(O)C(=O)C(O)C(O)CO]

and serve as a non-toxic functional replacement of the co-substrate in the DIOs. The mechanism of action of vitamin C at the different DIOs and the processes associated therewith was unclear up to now and can be explained by the function as a prodrug for DKG and 2-KGL. Thus, also a relatively broad activity of vitamin C at various target structures is caused, apart from redox effects [17, 49-61].

By using the modulators, there occurs a re-activation of the DIOs, and the competitive displacement of oncometabolites (hydroxyglutarate HG) from the active center is enabled. The function is recovered and adapted.

Examples for the influence of HGs and targets of the co-substrate regulation are summarized in Tables 1 and 2:

TABLE 1
Abnormous accumulation of D- and L-2-HG affects multiple cellular pathways

| 2-HG | Enzymes | Molecular | Affected cellular | Associated |
|---|---|---|---|---|
| enantiomer | generating HG | target | pathway | disease |
| D-2-HG | Mutant IDH1, | PHD/EGLN | HIF-1α | Glioma |
| | 2 | | | |
| D-2-HG | Mutant IDH1, | TET | DNA demethylation | Glioma, AML |
| | 2 | | | |
| D-2-HG | Mutant IDH1, | KDM | Histone demethylation | Glioma, AML |
| | 2 | | | |
| D-2-HG | Mutant IDH1 | ALKBH1, 2 | DNA repair | Glioma |
| D-2-HG | Mutant IDH2 | FTO | RNA demethylation | AML |
| D-2-HG | Mutant IDH2 | N.D. | ? | D-2HG azidurie |
| | | | | type II |
| D-2-HG | Mutant IDH1, | N.D. | STAT1 pathway; T | Tumor growth |
| | 2 | | cell function and | |
| | | | infiltration | |
| D-2-HG | Mutant IDH2 | N.D. | N.D. | Cardiomyopathy |
| D-2-HG | Mutant IDH1, | KDM4ADEPTOR | mTOR pathway | N.D. |
| | 2 | | | |
| D-2-HG | D2HGDH | N.D. | N.D. | D-2HG azidurie |
| | mutation | | | type I |
| D-2-HG | In vitro | | PIN1, NF-κB pathway | AML |
| | addition | | und stromal Zellen | |
| D-2-HG | In vitro | Cytochrome c | Cell respiration | |
| | addition | oxidase | | |
| L-2-HG | LDHAa | KDM | Hypoxia | L-2HG azidurie |
| L-2-HG | MDHa | KDM | Hypoxia | L-2HG azidurie |
| L-2-HG | L2HGDH | AASS | | L-2HG azidurie |
| | mutation | | | |
| L-2-HG | LDHA | KDM | T zell funktion and | Tumor |
| | | | infiltration | suppression |
| L-2-HG | L2HGDH low | N.D. | N.D. | Nierenkrebs |
| | expression | | | |

TABLE 2
Oncometabolite-affectable enzymes and associated tumors

| Enzyme | 2-HG enantiomer | Tumor |
|---|---|---|
| IDH1 | D-2HG | AML |
| | | Glioma |
| | | Secondary Glioblastomas |
| | | Chondrosarcoma |
| | | Cholangiocarcinoma |
| | | Melanoma |
| | | Prostate cancer |
| IDH2 | D-2HG | AML |
| | | Glioma |
| | | Secondary Glioblastomas |
| | | Chondrosarcoma |
| | | Cholangiocarcinoma |
| | | Angioimmunoblastic T-Cell Lymphomas |
| | | (Aitls) |
| PHGDH | D-2HG | Breast Cancer Cells |
| MYC | not specified | Breast Cancer |
| L2HGDH | L-2HG | Renal Cancer |

The use of the compounds according to the invention is also provided together with pharmaceutically applicable salts, tautomers and stereoisomers of the respective compound, including mixtures thereof.

The compounds represent a base for the further development and modification of the basic formulas. In the context of the present invention, pharmaceutically acceptable or applicable salts, prodrugs, enantiomers, diastereomers, racemic mixtures, crystalline forms, non-crystalline forms, amorphous forms, unsolvatized forms, and solvates of the general Formula (I) as disclosed.

The term “pharmaceutically applicable salts”, as used herein, includes salts of the compound according to the general Formulas (I) and (II) that are produced with relatively non-toxic (i.e., pharmaceutically acceptable) acids or bases depending on the special substituents found, for the compounds of the present invention. When, for example, the compounds of the present invention have acid functionalities, base addition salts can be obtained by contacting the neutral form of such compounds with a sufficient amount of the desired base, whether in a pure form or in a suitable inert solvent. Non-limiting examples for pharmaceutically acceptable base addition salts include sodium, potassium, calcium, ammonium, organic amino or magnesium salt or a similar salt. When compounds of the present invention have basic functionalities, acid addition salts can be obtained by contacting the neutral form of such compounds with a sufficient amount of the desired acid either in a pure form or in a suitable inert solvent. Non-limiting examples for pharmaceutically acceptable acid addition salts include such that are derived from inorganic acids, such as hydrochloric acid, hydrobromic acid, nitric acid, carbonic acid, phosphoric acid, partially neutralized phosphoric acids, sulfuric acid, partially neutralized sulfuric, hydroiodic or phosphoric acid and the like, as well as the salts that are derived from relatively non-toxic organic acids, such as acetic acid, propionic acid, isobuturic acid, maleic acid. Malonic, benzoic, succinic, su-ber, fumaric, mandelic, phthalic, benzenesulfonic, p-tolylsulfonic, citric, tartaric, methanesulfonic acid and the like. Also included are salts of amino acids, such as arginate and the like, and salts of organic acids, such as glucuronic or mucic acids and the like. Certain specific compounds of the present invention may have basic as well as acid functionalities that enable that the compounds are transformed either into base or acid addition salts. Contacting the salt with a base or acid can regenerate the neutral forms of the compounds of the present invention and isolate the parent compound in a conventional way. The original form of the compound differs from the different salt forms in certain physical properties, as solubility in polar solvents, however, other than that, the salts are, for the purposes of the present invention, equivalent to the original form of the compound. The compounds of the present invention may include chiral or asymmetric carbon atoms (optical centers) and/or double bonds. The racemates, diastereomers, geometric isomers, and individual optical isomers are comprised in the present invention. The compounds of the present invention may be present in unsolvatized forms as well as in solvatized forms, including hyperized forms. In general, the solvatized forms are equivalent to unsolvatized forms and are also included in the present invention. The compounds of the present invention may further exist in multiple crystalline or amorphous forms.

The compounds of the present invention may further exist in so-called prodrug forms. Prodrugs of the compounds of the invention are those compounds that easily undergo chemical changes under physiological conditions, in order to provide the compounds of the present invention. In addition, prodrugs in the compounds of the present invention can be transformed by chemical or biochemical methods in an ex-vivo environment. For example, prodrugs can slowly be transformed into the compounds of the present invention, when, for example, they are placed in a transdermal patch reservoir with a suitable enzyme or chemical reagent.

The compounds of the invention described herein can be administered to mammals, such as humans or domestic animals in a suitable dose. Non-limiting examples for domestic animals are pigs, cattle, buffaloes, sheep, goats, rabbits, horses, donkeys, chickens, ducks, cats, dogs, suids, or hamsters. Most preferably, it is administered to humans. The preferred kind of administration depends on the form of the compound of the invention (with the general Formula (I)). As described above, the compound with the general Formula (I) may be in the form of pharmaceutically acceptable salts, prodrugs, enantiomers, diastereomers, racemic mixtures, crystalline forms, non-crystalline forms, amorphous forms, non-solvatized forms or solvates. The compound of the invention can be administered orally, parenterally, such as subcutaneously, intraventrally, intramuscularly, intraperitoneally, intrathecally, intraocularly, transdermally, transmucosally, subdurally, locally, or topically via a iontophoresis, sublingually, by inhalation spray, aerosol or rectal and the like in dosage unit formulations that comprise, as appropriate, further conventional pharmaceutically acceptable excipients. The compound of the invention for use according to the present invention may be formulated as a pharmaceutical composition using one or more physiological carriers or excipients.

For oral administration, the pharmaceutical composition of the invention can assume, for example, the form of tablets or capsules that are prepared in a conventional way with pharmaceutically acceptable auxiliary substances such as binding agents (e.g., pre-gelated corn starch, polyvinylpyrrolidone, hydroxypropylmethylcellulose), filling agents (e.g., lactose, microcrystalline cellulose, calcium hydrogen phosphate), sliding agents (e.g., magnesium stearate, talcum, silicium dioxide), disintegration agents (e.g., pota starch, sodium starch glycolate), or wetting agents (e.g., sodium lauryl sulfate). The pharmaceutical composition can be administered to a patient with a physiologically acceptable carrier. In a specific embodiment, the term “pharmaceutically acceptable” means that it is approved by a regulation authority or another generally accepted pharmacopoeia for use in animals and in particular in humans. The term “carrier” relates to a diluent, adjuvant, excipient or vehicle, with which the therapeutic is administered. Such pharmaceutical carriers may be sterile liquids, such as water and oils, including those of petroleum, animal, vegetable or synthetic origin, such as peanut oil, soybean oil, mineral oil, sesame oil, and the like. Water is a preferred carrier, when the pharmaceutical composition is administered intravenously. Salt solution and aqueous dextrose and glycerol solutions can also be used as liquid carriers, in particular for injectable solutions. Suitable pharmaceutical auxiliary substances include starch, glucose, lactose, sucrose, gelatin, malt, rice, flour, chalk, silica gel, sodium stearate, glycerol monostearate, talcum, sodium ion, dried skimmed milk, glycerin, propylene, glycol, water, ethanol, and the like. If desired, the composition may also contain small amounts of wetting or emulsifying agents or pH buffering agents. These compositions may be in the form of ointments, solutions, suspensions, emulsions, tablets, pills, capsules, powders, sustained-release formulations, and the like. A preferred form is an ointment. The composition can be formulated as a suppository with traditional binding agents and carriers such as triglycerides. The oral formulation may contain standard carriers, such as mannitol, lactose, starch, magnesium stearate, sodium saccharine, cellulose, magnesium carbonate, etc. of pharmaceutical grade. E. W. Martin describes examples of suitable pharmaceutical carriers in “Remington's Pharmaceutical Sciences”. Such compositions contain a therapeutically effective amount of the aforementioned compounds, preferably in a purified form, together with a suitable amount of carrier so as to provide the form for proper administration to the patient. The formulation should correspond to the route of administration. Liquid preparations for the oral administration may be in the form of, for example, solutions, syrups, or suspensions, or may be presented as a dry product for use with water or another suitable vehicle before use. Such a liquid preparation may be formulated in a conventional manner with pharmaceutically acceptable additives such as suspending agents (e.g., sorbitol, syrup, cellulose derivatives, hydrogenated edible fats), emulsifiers (e.g., lecithin, acacia), non-aqueous vehicles (e.g., almond oil), oil, oily esters, ethyl alcohol, fractionated vegetable oils), preservation agents (e.g., methyl or propyl-p-hydroxycarbonates, sucrose acids). If appropriate, the preparations may also contain buffer salts, flavoring, coloring, and sweetening agents. Preparations for oral administration may suitably be formulated to allow a controlled release of the pharmaceutical composition of the invention.

For administration by inhalation, the pharmaceutical composition of the invention is conveniently delivered in the form of an aerosol spray presentation from a pressurized pack or a nebulizer using a suitable propellant (e.g., dichlorodifluoromethane, trichlorofluoromethane, dichlorotetrafluoroethane, carbon dioxide or another suitable gas). In the case of a pre-dispersed aerosol, the dosage unit may be determined by providing a valve to deliver a metered amount. Capsule and cartridges of, for example, gelatin for use in an inhalator or insufflator may be formulated containing a powder mixture of the pharmaceutical composition of the invention and a suitable powder base, such as lactose or starch.

The pharmaceutical composition of the invention may be formulated for parenteral administration by injection, for example by bolus injection or continuous infusion. The site of the injection is intravensal, intraperitoneal, or subcutaneous. Formulations for injection may be presented in unit dosage forms (e.g., in vials, in multi-dose containers) and with an additional preservation agent. The pharmaceutical composition of the invention may take such forms as suspensions, solutions, or emulsions in oily or aqueous vehicles and may contain formulating agents, such as, for example, suspending, stabilizing, or dispersing agents. Alternatively, the agent may be in powder form for constitution with a suitable vehicle (e.g., sterile pyrogen-free water) prior to use. Typically, the compositions for intravenous administration are solutions in sterile isotonic aqueous buffers. If necessary, the composition may also contain a solubilizing agent and a local anesthetic, such as lignocaine, so to relieve pain at the injection site. In general, the ingredients will be mixed together either separately or in unit dosage form, for example as a dry lyophilized powder or anhydrous concentrate in a hermetically sealed container, such as a vial or a bag indicating the amount of active agent. When the composition is to be administered by infusion, an infusion bottle containing sterile water or a pharmaceutical-grade salt may be dispensed with. When the composition is administered by injection, a vial with sterile water for injection or saline may be provided so that the ingredients may be mixed prior to administration.

For an average person skilled in the art, it will be apparent that the present invention also includes dosage forms with sustained release designed to release a drug at a predetermined rate, in order to maintain a constant drug concentration for a given period of time with minimal side effects. This can be accomplished by numerous formulations or devices, including microspheres, nanoparticles, liposomes and other polymer matrices, such as drug-polymer conjugates such as hydrogels or biologically degradable substances such as poly(lactic co-glycolic acid) (PLGA), which encapsulate the active ingredient. It is preferred to adapt the release to the specific needs for the treatment of certain diseases, such as e.g., the sustained release of injections in the treatment of diabetes. The definition of sustained release is more similar to a “controlled release” or “depot medication” than a “sustained” one.

The pharmaceutical composition of the invention may also be provided, if desired, in a package or a donor that may contain one or more unit dosage forms containing the agent. The package may include, for example, a metal or plastic film, such as a blister pack. The pack or dispenser device may be accompanied by instructions for administration.

The pharmaceutical composition of the invention may be administered as the sole active ingredient or in combination with other active ingredients. Such additional active agents should be selected primarily from active ingredients that are associated with the treatment of the same disease. In the case that obesity is to be treated, an additional active ingredient should be selected from the group of anti-obesity drugs. Analogously, anti-diabetic drugs as well as anti-NAFLD/NASH and anti-dyslipidemic drugs are used as further active ingredients. Furthermore, such an additional active ingredient should be selected from active ingredients that are associated with side effects such as body weight gain such as anti-psychotive treatments.

The compounds according to the invention or the compositions according to the invention may be used as co-substrates for the prevention, treatment, and after-treatment of tumor diseases. Preferably, the tumor disease is a disease selected from the group including tumors of the neck-nose-ears region, including tumors of the inner nose, paranasal sinuses, nasopharynx, lips, oral cavity, oropharynx, larynx, hypopharynx, ear, salivary glands, and paragangliomas, growths in the lung, including non-parvicellular bronchial carcinomas, parvicellular bronchial carcinomas, tumors of the mediastinum, tumors of the gastrointestinal tract, including tumors of the esophagus, of the stomach, the pancreas, the liver, the gallbladder and the biliary tracts, small intestine and intestine carcinomas and anal carcinomas, urogenital tumors, including tumors of the kidneys, ureter, bladder, prostate gland, penis and testicles, gynecological tumors including tumors of the cervix, vagina, vulva, uterus cancer, malignant trophoblastic disease, ovarian carcinoma, uterine tubes (Fallopian tubes), tumors the abdominal cavity, mammary carcinomas, tumors of the endocrine organs, including tumors of the thyroid, parathyroid, adrenal cortex, endocrine pancreas tumors, carcinoids and carcinoid syndrome, multiple endocrine neoplasias, bone and soft part sarcomas, mesotheliomas, skin tumors, melanomas from cutaneous and intraocular melanomas, tumors of the central nervous system, tumors in infancy, including retinoblastoma, Wilm's tumor, neurofibromatosis, neuroblastoma, Ewing's sarcoma tumor family, rhabdomyosarcoma, lymphomas including non-Hodgkin lymphomas including cutaneous T-cell lymphomas, primary lymphomas of the central nervous system, morbus Hodgkin, leukemias including acute leukemias, chronic myelogenous and lymphatic leukemias, plasma cell neoplasmas, myelodysplasia syndrome, paraneoplastic syndromes, metastases with unknown primary tumor (CUP syndrome), metastasizing tumors including brain metastases, lung metastases, liver metastases, bone metastases, pleura and pericardial metastases and malignant ascites, peritoneal carcinosis, immunosuppression-induced malignity including AIDS-associated malignity, such as Kaposi's sarcoma, AIDS-associated lymphomas, AIDS-associated lymphomas of the central nervous system, AIDS-associated morbus Hodgkin and AIDS-associated anogenital tumors, transplant-related malignity.

In the context of the treatment of tumor diseases, the compounds according to the invention are combined with chemotherapeutics, which may be selected from the group including antibodies, alkylation agents, platinum analogs, intercalation agents, antibiotics, mitose suppressors, taxanes, topoisomerase suppressors, antimetabolites and/or L-asparaginase, hydroxycarbamide, mitotane and/or amanitine.

Further aspects, features, and advantages of the present invention readily follow from the following detailed description, in which preferred embodiments and implementations are explained. The present invention may also be implemented in other and different embodiments, and its various details can be modified in different, apparent aspects, without departing from the teaching and scope of the present invention. Correspondingly, the drawings and descriptions are to be considered as illustrating and not as limiting. Additional objects and advantages of the invention are partially outlined in the following description and become partially apparent from the description or can be taken from the execution of the invention.

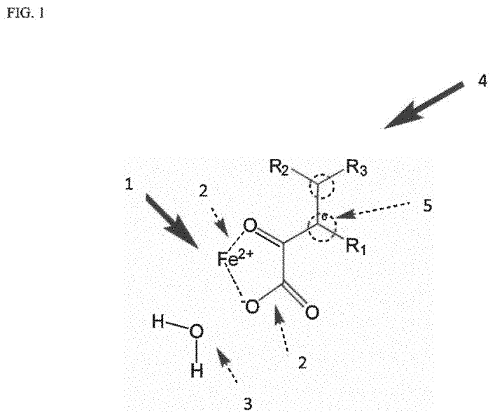

FIG. 1 shows the binding conditions in the enzyme being necessary for the function as a co-substrate. For this purpose, a divalent iron atom 1, which does not covalently interact with the co-substrate, is in the center of reaction of the enzyme. The necessary distance co-substrate/iron is in the range of 2.1-2.4 Å (arrow 2). The water molecule has a distance of 2.3 Å (arrow 3) and plays an important role in the reaction in progress. Altogether, the transition state (TS) for the reaction in the enzyme of the co-substrate or the functional replacement of co-substrates is shown here. Furthermore, FIG. 1 shows the environment in the active center of DIOs and the embedment and the non-covalent fixation of the co-substrate/co-factor complex 4 (cf. also 2-His-1-carboxylate facial triad [41] [42]). Further is shown a carbon atom with identical electronic properties 5.

The compounds of the present invention according to Formula (I) and (11) can actively or passively arrive in the target cell via transporters (e.g., DKA, 2-OKG) or after the corresponding derivatization.

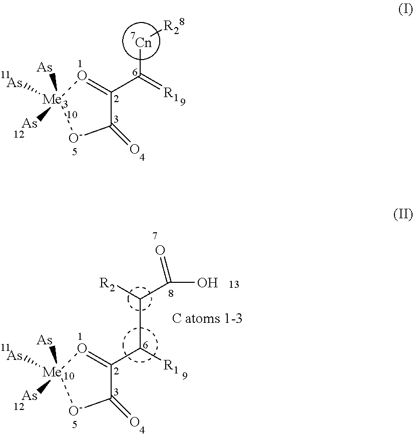

wherein
As represent the amino acids from the binding pocket of the enzyme; Me represents a metal from the catalytic center;
R₁ and R₂ may be oxygen (hydroxyl) or carboxyl groups, halogens, in particular fluorine, chlorine, or iodine, a mono- or poly-halogenated methyl group, in particular, CH₂F up to CF₃; and
Cn represents a C atom, a heteroatom, or the bridge to a heterocycle.

This procedure of using functional equivalents of the physiological co-substrate 2-OG in DIOs is unknown up to now and in total represents a paradigm change in the functional influence and recovery of human metabolic enzymes for the treatment of diseases. It is delimited, thus, from other options that are aimed at affecting the catalytic metal (co-factor) of the enzyme or reducing the formation of oncometabolites such as 2-HG (so-called IDH inhibitors). Thus, this paradigm change is expressly notable. By this new method and the demonstrated efficiency thereof, previously open questions of the functionality of DIOs such as the ten-eleven translocation (TET) enzymes and HIF prolinhydroxylases become explainable [21].

The present invention is based on the discovery of compounds, which recover the function of DIOs also in presence of oncometabolites (HG), and thereby comprehensive possibilities of treatment of diseases become available. Up to now, no compounds have been described that can mimic the physiological function of the 2-OG as a co-substrate.

On the one hand, it is a completely new approach of affecting enzymes in living cells, and on the other hand, the present invention enables to answer extensively and for the first time open question with regard to DIOs, and to derive new therapeutic options for DIO-dependent diseases therefrom.

The present invention is further based on docking experiments and molecular-dynamic investigations with methods and algorithms according to Homann, whereby it became possible to show the actual conditions in the active site (binding or interaction site) of the DIO enzyme and to be able to prove them by corresponding experiments in cell-free and cell systems.

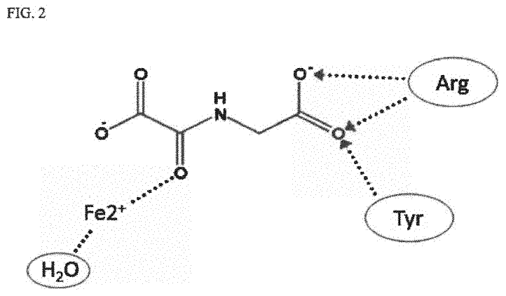

Thus, from X-ray structure investigations of the TET2 enzyme, the binding of the TET inhibitor NGA (N-oxalylglycine) is known (cf. FIG. 2).

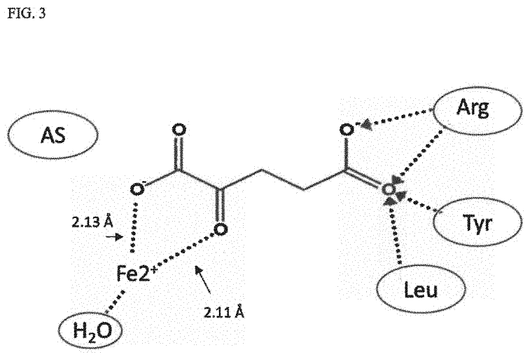

The binding conditions of 2-OG in the TET enzyme were previously not known and were assumed in analogy to FIG. 2. In FIG. 3 and FIG. 4, these binding conditions (in the TS) are shown for the first time. Based on FIG. 1 and FIG. 2, it can be seen that the binding distances necessary for the enzyme reaction are maintained. Water is, as already explained in FIG. 1, a necessary component in the enzyme complex and is shown here, too.

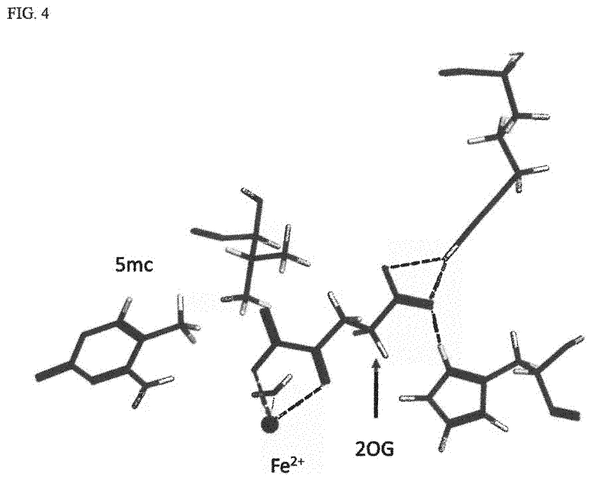

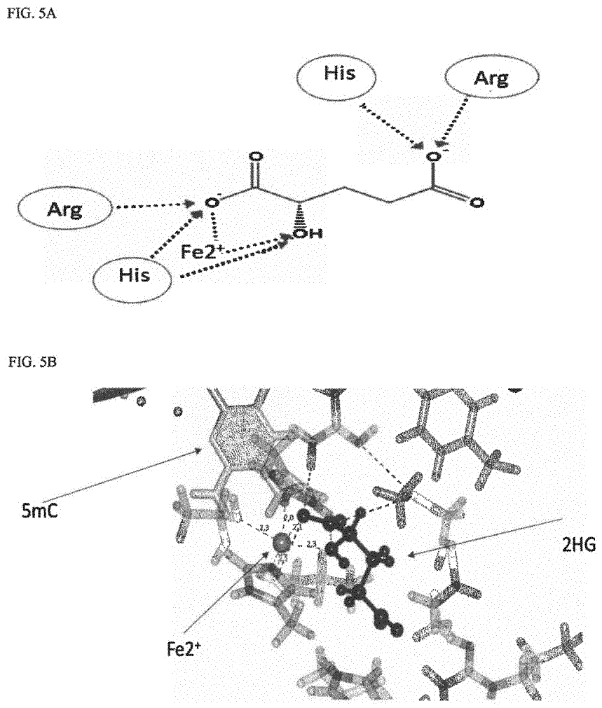

In order to be able to generally explain the function loss of DIOs (here with respect to the example of TET) by the oncometabolite 2-HG, its spatial structure in the enzyme had also to be clarified (cf. FIG. 4). Here, we can show how 2-HG interacts in the TET and HIF enzyme, and by the interaction of the hydroxyl group with the catalytic metal (Fe2⁺), it cannot enter into the reaction as a co-substrate. At the same time, by the stronger affinity of the 2-HG in the enzyme, 2-OG is competitively displaced. Thus, the enzyme becomes non-functional (FIG. 5A and FIG. 5B).

Based on the investigations and results mentioned above, it was found that a chemical compound that is to have the functionality of the 2-OG, has to be in the same kind in the enzyme under similar electronic conditions (FIG. 1).

From these first results, it emerged that for the functional replacement of the 2-OG and for the recovery of the DIO function, only such compounds can be contemplated that bind in the DIO enzyme stronger than 2-OG, but at the same time can have and accept the functionality as a co-substrate. A pure stronger binding alone would lead to an inhibition of the DIOs that then would be similar to that of the 2-HGs and leads, same as 2-HG, to a competitive inhibition [43]. This is counterproductive for the desired function as a functional equivalent.

If, however, the stronger binding in the DIO enzyme occurs as a functional equivalent that enters into the corresponding reactions, then compounds are present that can displace 2-HG and recover the DIOs by the co-substrate function (e.g., TET function of the demethylation of 5-methylcytosine 5mC). These compounds alone are capable to replace the 2-OG.

The functional replacement of the co-substrate 2-OG by DKA is shown in FIG. 6, which shows the results of a cell-free assay for the function of DKA in the TET enzyme as a functional co-factor. For this purpose, the investigations were made in the cell-free TET assay (in 50 mM HEPES buffer with 50 mM NaCl 8 μM Fe(NH₄)₂(SO₄)₂ 5 mM ATP, 3 mM DTT, 0.25 μg TET enzyme, and herring sperm DNA. After 8-hour incubation, the evaluation was performed by means of mass spectrometry. The native 2-OG was replaced by the functional co-enzyme DKA. It could be shown that DKA can assume the function of the native 2-OG and serve as a replacement of the native co-substrate.

Mutations in the genes coding the isocitrate dehydrogenase (IDH) require the reduction of 2-OG to the oncometabolites D-2-hydroxyglutarate (2-HG) leading to an inhibition of the demethylation of 5-methylcytosine (5mC) in the DNA to 5-hydroxymethylcytosine (5hmC) by (TET), and to methylated histone lysine (HK) residues (HKme) by Jumonji C-domain demethylases (JMJC) and N6-methyladenosine (m6A) by FTO [44, 45].

2-HG is an oncometabolite inhibiting numerous demethylases, which leads to changes in genomic and transcriptional methylation profiles and to changes in gene expression and genome topology [46]. Therefrom result corresponding symptoms, inter alia, cancer and when regarding the total methylation of the genome, aging processes.

The cornerstone of cancer therapy, including the later success of epigenetic therapies, is the use of effective and rational drug combinations. In the focus is the combination of epigenetically effective drugs (also included are here our 2-OG functional replacement co-substrates) with other therapies and the optimization of the same.

Insofar, the TET enzymes represent, due to their central epigenetic role, a special target for the replacement of the native co-enzyme 2-OG.

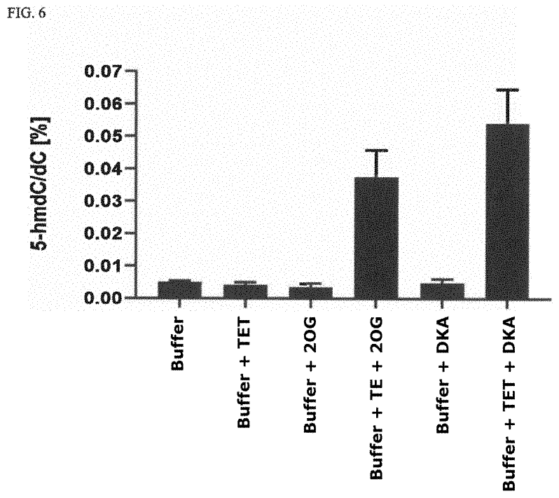

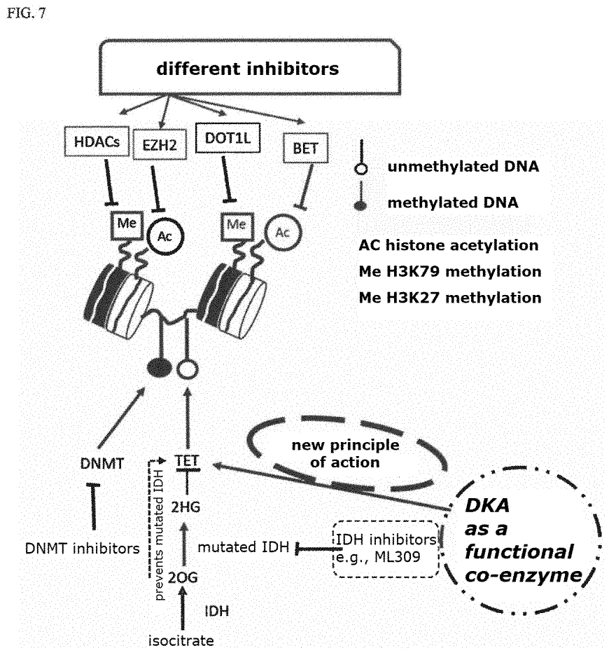

In the following, the use of the compounds according to the present invention is described for the TET enzyme. The previously known scientific literature is based, when TET enzymes are regarded as targets for therapies, on the situation illustrated in FIG. 7.

In FIG. 7, known therapies are shown, complemented by the new pharmacological concept according to the present invention. The abbreviations have the following meanings: HDAC—histone deacetylases; EZH2—enhancers of zeste homolog 2, a histone-lysine N-methyltransferase enzyme; DOT1L—disruptor of telomeric silencing 1-like; BET—bromodomain and extra-terminal motif.

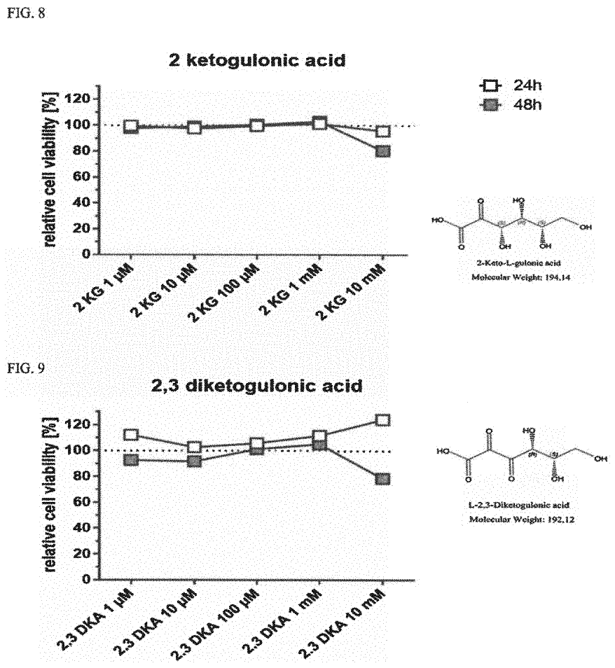

A functional replacement of the co-substrate and the function takeover by an externally supplied compound has never been considered in prior art and thus represents a paradigm change. The compounds according to the present invention, in particular the DKA and the 2-ketogulonic acid (2-OG), can be used due to their non-toxic effect, also in the corresponding pharmacological concentrations (highest used concentration is 1 mM), as indicated in FIG. 8 and FIG. 9, which show the results of investigations about the toxicity.

In clinical studies, at present, different combination strategies are examined, for example, the combination of epigenetic therapies with chemotherapy, targeted therapies, and immunotherapies. Functional equivalents of the 2-OG in TETs could heretofore not be considered in combination therapies, since they were unknown to date and are only contemplated with the present invention.

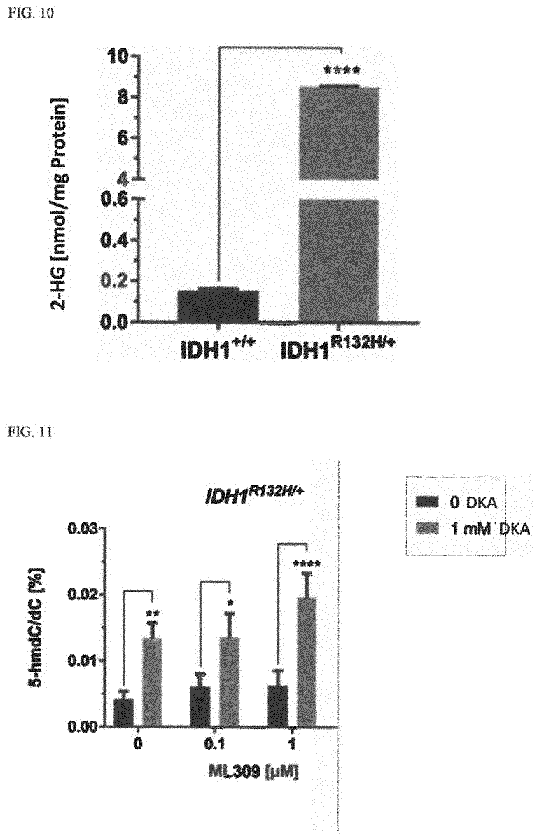

The detection that cancer cells can escape from the selective pressure by transcriptional adaptation, provides a molecular explanation for the use of the epigenetic therapy blocking or reversing the resistance. In IDH inhibitors that are right now in first clinical studies, this is also shown [47]. By the combination of IDH inhibitors with co-substrates according to the present invention, a potentiation of the effectivity of the IDH inhibitors can be demonstrated on the cell level (cf. FIG. 10), whereby the potential of the new compounds according to the present invention in the environment of a new enzyme affectation is impressively shown.

Likewise, promotion of the secondary apoptosis occurs, which indicates the recovery of the cell function on a molecular level (cf. FIG. 11). The native 2-OG again enters into the normal cell metabolism and thus again inhibits the malignant derailment of the cell (secondary way of formation of 2-OG from glutamic acid is omitted). The incubation with the strong IDH inhibitor ML309 that significantly reduced the 2-HG level, does not activate the inhibited TET enzymes in IDH1R132H/+ cells. By the addition of the functional co-substrate DKA, activation of the TET enzyme occurred.

Another fact that proves the advantages of compounds according to the present invention replacing the native co-substrate 2-OG, is that thereby new compounds can be designed that are also active at mutated DIOs and differ in the kinetics and binding affinity from the natural co-substrate.

Es is known that IDH1 mutations are connected with a modified IDH1 enzyme function that induces the excess production of neomorphous metabolite 2-hydroxyglutarate. In order to validate the increased frequency of 2-HG in HCT116 IDH1R132H/+ cells compared to HCT116 IDH1+/+ cells, the 2-HG content was analyzed by means of LC-MS/MS analysis (illustration 1). Intracellular 2-HG in IDH1R132H/+ cells was 56 times higher than in IDH1 wildtype cells (8.5 nmol/mg protein or 0.15 nmol/mg protein; 56 times higher; p<0.0001). Furthermore, the treatment with the IDH1 inhibitor ML309 (10 μM) led to a significant reduction of the oncometabolite 2-HG in the HCT116 IDH1R132H/+ cells to (0.3 nmol/mg protein) and to a lower degree of IDH1+/+ (0.04 nmol/mg protein) (Fig.). In contrast, DKA produced only minimum changes in the 2-HG concentrations in the mutated cells (5.9 nmol/mg protein). Interestingly, the combinatory treatment with 10 μM ML309 and 1 mM DKA led to the strongest reduction of 2-HG for IDH1R132H/+ and reached a similar concentration as for the IDH1-wildtype cells (0.12 nmol/mg protein), cf. FIG. 10.

FIG. 11 shows that 2-hydroxyglutarate levels correlate with 5-hmdC levels in IDH1-MT HCT116.

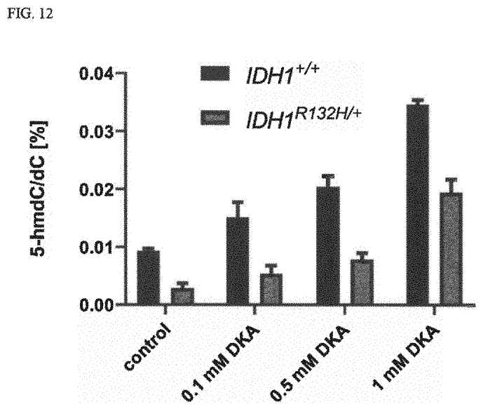

FIG. 12 shows that by the use of DKA alone as a functional co-substrate, the function of the TET enzyme can also be recovered in presence of the competitive inhibitor 2-HG.

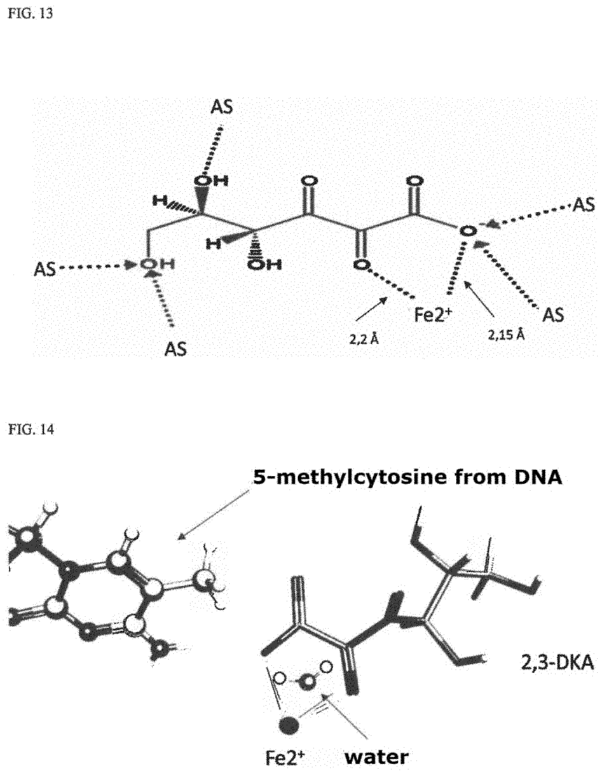

In Figures FIG. 13-18, further selected chemical compounds that can act as functional replacements of the co-substrate 2-OG at TET and HIF, are shown in their conformation in the enzyme. In this context, corresponding cell experiments are listed further below.

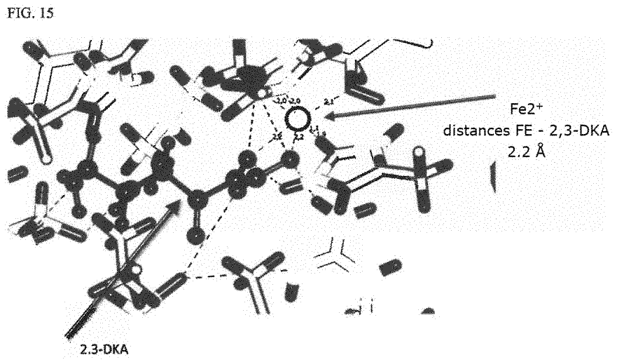

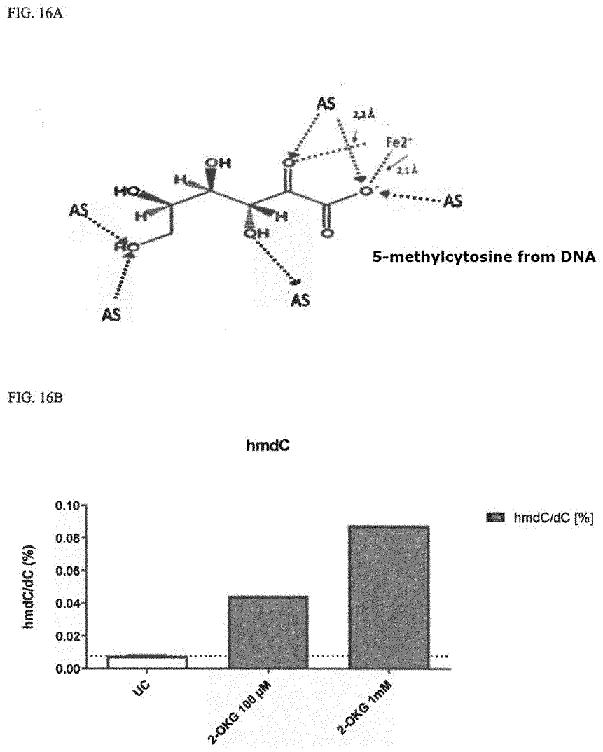

FIG. 13 shows the TET enzyme DKA, and indicates to which atoms amino acids AS coordinate. In FIG. 14, DKA is shown in a spatial structure, and in FIG. 15 is shown the spatial coordination of DKA in the enzyme. The arrow in FIG. 15 indicates the distance of the Fe2⁺ from DKA with 2.2 Å. It is shown that DKA is located in the TS in the enzyme, and the given conditions (FIG. 1) are here also achieved so that a functional replacement of the co-substrate 2-OG is enabled.

FIG. 16A shows 2-ketogulonic acid (2-OKG) and shows how the 2-OKG is located in the TS in the enzyme, and the given conditions (FIG. 1) are here also achieved, so that a functional replacement of the co-substrate 2-OG is enabled. In FIG. 16B is shown, in the assay, that 2-OGK has a dose-dependent effect.

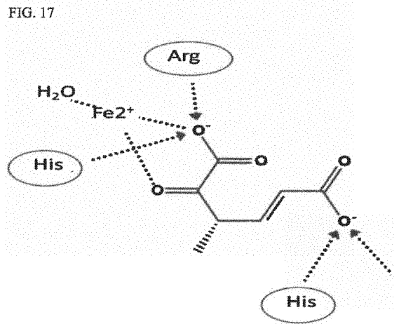

FIG. 17 shows 4-methyl-5-oxohex-2-enedioic acid with Fe2⁺ under indication of the amino acids that interact.

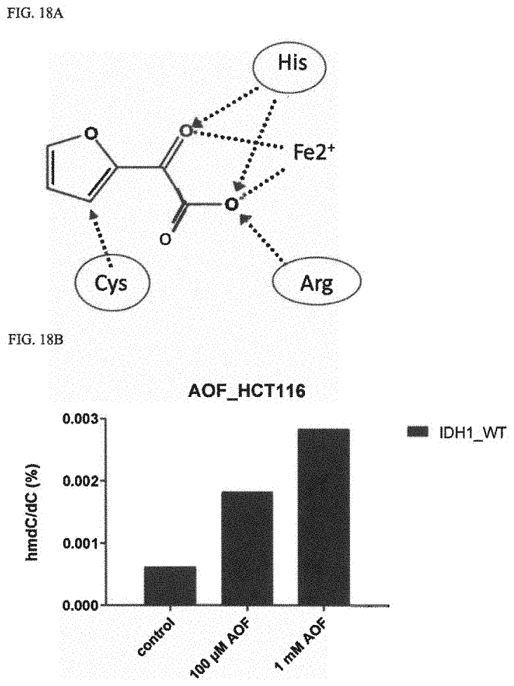

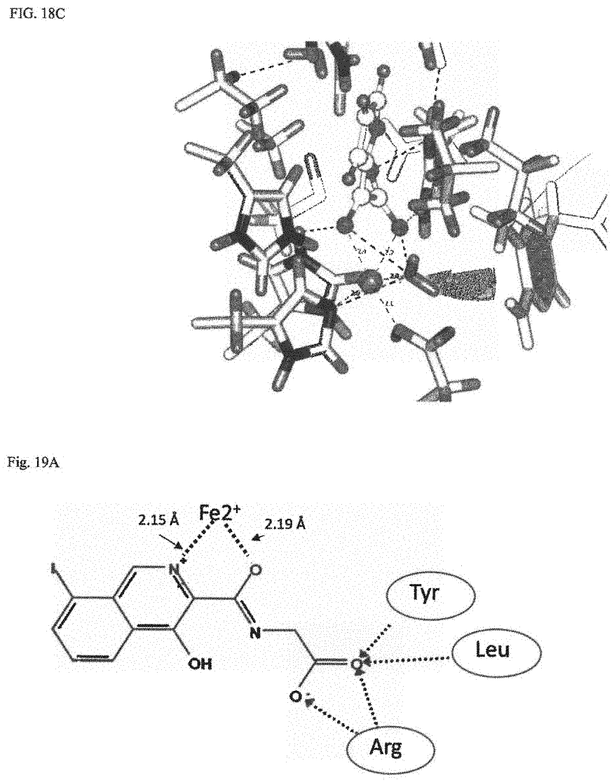

In FIG. 18A is shown AOF (2-(furan-2-yl)-2-oxoacetate with Fe2⁺ and amino acids, in FIG. 18B is shown the effect, and in FIG. 18C is shown the interaction between enzyme and AOF.

FIG. 19-27 shows compounds for the HF enzyme. In FIG. 19A, the structure is shown, and in FIG. 19B, in 2G19, 2-((hydroxy(4-hydroxy-8-iodoisoquinoline-2-ium-3-yl)methylene)amino)acetate is bound in a hypoxia-induced factor (hypoxia-inducible factor prolyl hydroxylase, PHD2) as an antagonist.

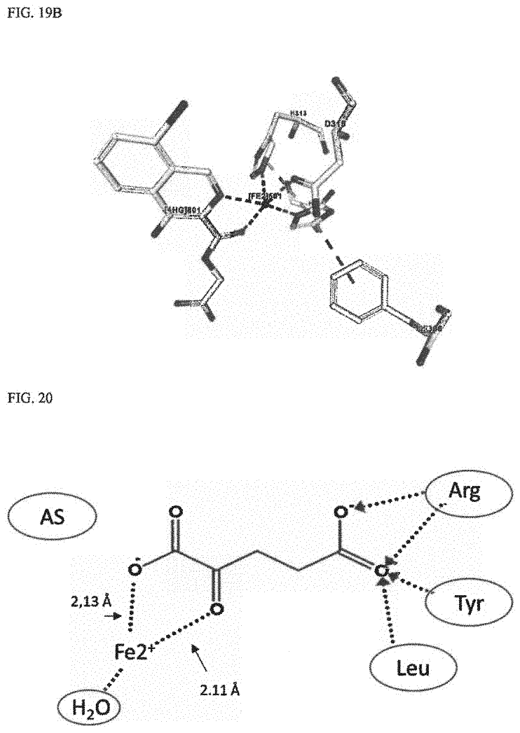

In FIG. 20, 2-OG is shown in the enzyme PHD2 (according to method Homann). The position in the enzyme corresponds to the necessary principle for the function as a co-substrate.

In FIG. 21, NGA is shown as an antagonist in the enzyme.

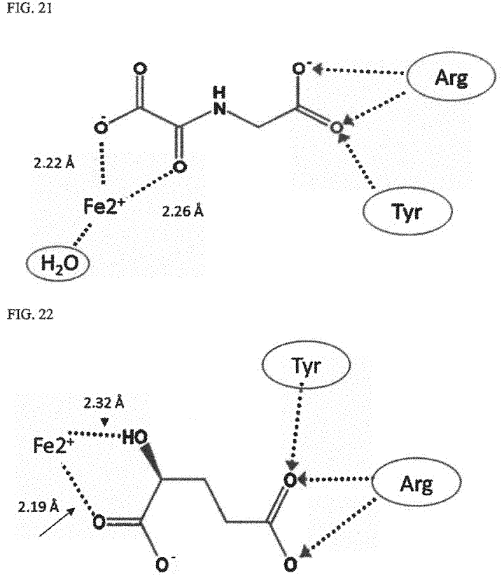

FIG. 22 shows the competitive antagonist 2-HG in the enzyme that competitively interacts in the enzyme with 2-OG and leads to the function loss of the enzyme.

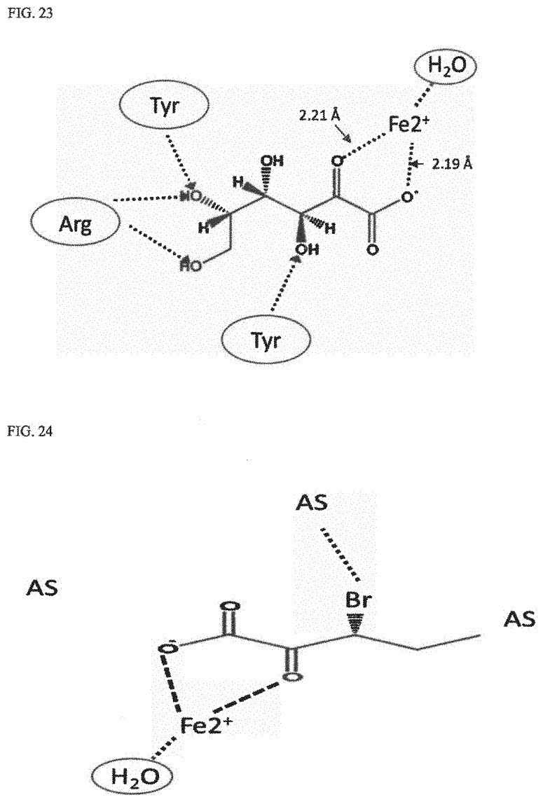

FIG. 23 shows 2-OKG in the enzyme as a functional co-enzyme. The necessary principles for this function are maintained.

FIG. 24 shows 3-bromo-2-oxopentanoate in the enzyme as a functional co-enzyme.

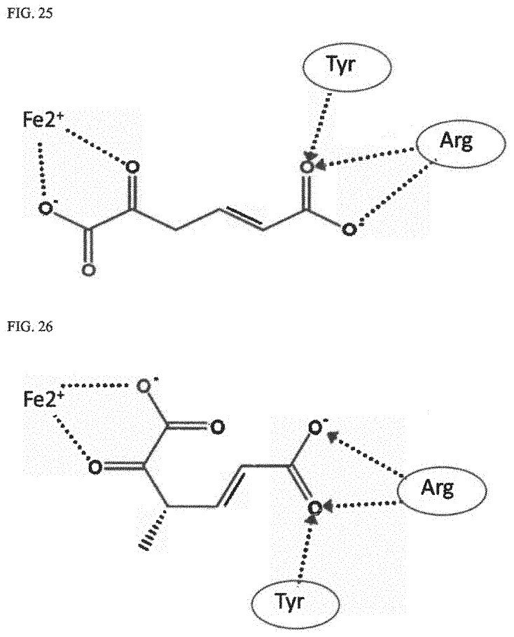

FIG. 25 shows (E)-5-oxohex-2-enedioic acid in the enzyme as a functional co-enzyme.

FIG. 26 shows 4-(S)-methyl-5-oxohex-2-enedioic acid in the enzyme as a functional co-enzyme.

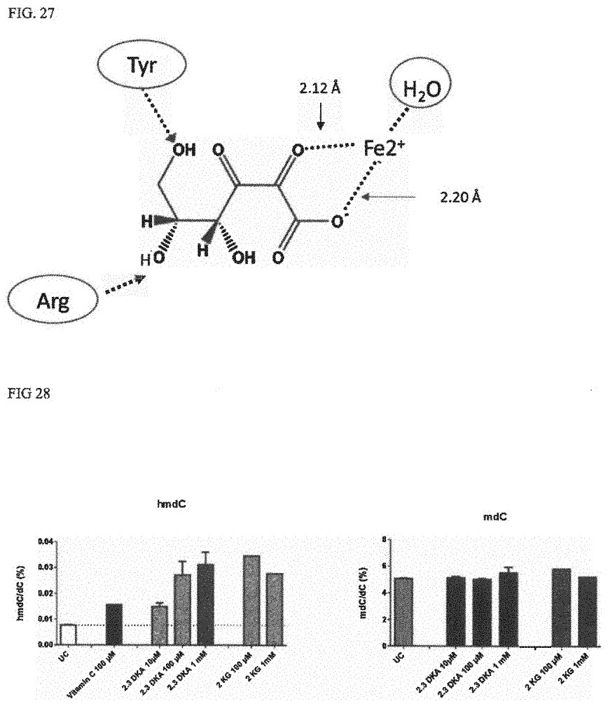

FIG. 27 shows DKA in the enzyme as a functional co-enzyme.

### EMBODIMENTS

### TET (Ten-Eleven Translocation) Enzymes

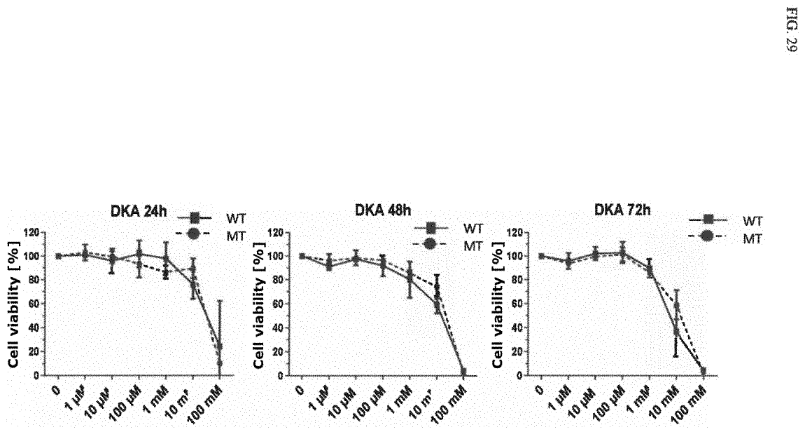

In Example 1 (FIG. 28) are shown the recovery of functionality, and how epigenetic changes leading to the occurrence of cancer are reversed.

Furthermore, the regeneration of the normal metabolism of the cell is possible, and the cancer metabolism is interrupted. This leads, in connection with other chemotherapies (combination therapy) or alone to a normalization of the cell function and the possibility of the apoptosis that is prevented in degenerated cells. The re-activation of tumor suppressor genes (CDKN1A (p21)) also occurs by the again functionable TETs.

FIG. 28 shows DKA-2,3-diketogulonic acid from Example 1 and 2-OG (2-KG-2-keto-L-gulonic acid):

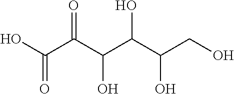 [SMILES: O=C(O)C(=O)C(O)C(O)C(O)CO]

### Cell Culture and Treatment

HCT116 (ATCC-Nr. CCL-247), a human colorectal carcinoma cell line, was acquired from the American Type Culture Collection (ATCC; https://www.atcc.org/). Human colon carcinoma cells HCT116 IDH1+/+ and HCT116 IDH1R132H/+ come from Horizon Discovery and were let us for test purposes. The cells were cultured in Dulbecco's Modified Eagle's Medium (DMEM) with 2 mM L-glutamine, supplemented with 10% fetal bovine serum (FBS), 45 IU/ml penicillin and 45 IU/ml streptomycin. The cell lines were negatively tested for mycoplasma infections within six months before use.

### Test of Cell Viability

The investigation of the possible cytotoxic effects of the tested substances was performed with the MTT reduction assay, as already described. HCT116 cells were placed into 96-well plates (TPP, Trasadingen, Switzerland). After 24 h, the mentioned concentrations were added for 24, 48, and 72 h. Then, the cells were incubated with 100 μL MTT solution (0.5 mg/ml in PBS) for 4 h. After removal of the supernatants, 50 μL dimethyl sulfoxide were added to dissolve the formazon salt, and the optical density (OD) was measured with a microplate reader (Tecan, Crailsheim, Germany). The excitation was set to 540 nm. The positive controls were treated with 0.002% SDS. A cell viability <75% indicates cytotoxic effects.

### Apoptosis Assay

The level of apoptotic and dead cells was determined by means of flow cytometry using the eBiosciencem Annexin V Apoptosis Detection Kit APC (Thermo Fisher, Darmstadt, Germany). The cells were sown 2×10⁵ HCT116 cells/well in 6-well plates (TPP, Trasadingen, Switzerland). After 24 h, the cells were incubated with the substances in the indicated concentrations for 72 h. Then, the cells were washed and stained with Annexin V and propidium iodide as per the instructions of the manufacturer. The cells were analyzed on a FACSCanto 11 (BD Biosciences, Heidelberg, Germany). For the data analysis, the software FlowJo (Treestar, Ashland, USA) was used.

### RT-PCR

The RNA was extracted according to the instructions of the RNA High Pure RNA Kit (Roche, Mannheim, Germany), and 0.5-5 μg (ideally 3 μg) of the RNA was reverse-transcribed with the aid of the RevertAid Reverse Transcriptase (Thermo Fisher, Darmstadt, Germany) according to the protocol. The qRT-PCR was performed with the Maxima SYBR Green qPCR Mix (ThermoFisher, Darmstadt, Germany) on a Lightcycler 480 II Real-Time PCR System (Roche, Mannheim, Germany). The quantification was performed with the method AA Ct, and the GAPDH print was used as an internal reference. The analysis of the melting curve confirmed that all qRT-PCR products were generated in the form of double-stranded DNA. The used primers are listed in Table 3.

TABLE 3
Determination of the genome-wide DNA methylation and
hydroxy methylation by means of isotope dilution, liquid
chromatography, tandem-mass spectrometry (LC-MS/MS)

| | | | Fragment Size |
|---|---|---|---|
| Target gene | Sequence | | (bp) |
| HHMBS | fw: | ACCAAGGAGCTTGAACATGC (SEQ ID NO: 1) | 143 |
| | rv: | GAAAGACAACAGCATCATGAG (SEQ ID NO: 2) | |
| hDNMT1 | fw: | ACCTGGCTAAAGTCAAATCC (SEQ ID NO: 3) | 80 |
| | rv: | ATTCACTTCCCGGTTGTAAG (SEQ ID NO: 4) | |
| hDNMT3a | fw: | ACTACATCAGCAAGCGCAAG (SEQ ID NO: 5) | 359 |
| | rv: | CATCCACCAAGACACAATGC (SEQ ID NO: 6) | |
| hDNMT3b | fw: | CCAGCTCTTACCTTACCATC (SEQ ID NO: 7) | 285 |
| | rv: | CAGACATAGCCTGTCGCTTG (SEQ ID NO: 8) | |
| hETE1 | fw: | GCTGCTGTCAGGGAAATCAT (SEQ ID NO: 9) | 209 |
| | rv: | ACCATCACAGCAGTTGGACA (SEQ ID NO: 10) | |
| hTET2 | fw: | CCAATAGGACATGATCCAGG (SEQ ID NO: 11) | 232 |
| | rv: | TCTGGATGAGCTCTCTCAGG (SEQ ID NO: 12) | |
| hTET3 | fw: | TCGGAGACACCCTCTACCAG (SEQ ID NO: 13) | 179 |
| | rv: | CTTGCAGCCGTTGAAGTACA (SEQ ID NO: 14) | |
| CDKN2A | fw: | GAGCAGCATGGAGCCTTC (SEQ ID NO: 15) | 124 |
| (p16) | rv: | CCTCCGACCGTAACTATTCG (SEQ ID NO: 16) | |
| CDKN1A | fw: | AGTGGACAGCGAGCAGCTGA (SEQ ID NO: 17) | 381 |
| (p21) | rv: | TAGAAATCTGTCATGCTGGTCTG (SEQ ID NO: 18) | |
| CDKN1B | fw: | AAACGTGCGAGTGTCTAACGGGA (SEQ ID NO: 19) | 456 |
| (p27) | rv: | CGCTTCCTTATTCCTGCGCATTG (SEQ ID NO: 20) | |

Samples of genomic DNA (20 μg) were hydrolyzed with micrococcal nuclease from Staphylococcus aureus, bovine spleen phosphodiesterase, and bovine intestinal alkaline phosphatase (all from Sigma-Aldrich, Taufkirchen, Germany) to 2′-deoxynucleosides, as described [62], with modifications of the application. 10 μL of 50 nM 5-hmdC-d3 (Toronto Research Chemicals, Toronto, Canada) were added as an internal standard to the DNA reaction mixture, and the incubation time of the two-step hydrolysis was 1 h each. Then, DNA hydrolysates were centrifuged (5 min, 16,000×g), and 10 μL of the supernatants were used for the quantification of dC and 5-mdC stable marked references.

Compounds [15N2,13Cl]dC and 5-mdC-d3 (both from Toronto Research Chemicals, Toronto, Canada). The residues of the DNA hydrolysates (˜310 μL) were evaporated with a Savant SpeedVac Concentrator (Thermo Fisher Scientific, Dreieich, Germany) under reduced pressure to dryness. After the addition of 100 μL methanol to the dried residues and short vortexing, the samples were stored overnight at −20° C. On the next day, the samples were thoroughly vortexed for 10 minutes (1,400 rpm) and then centrifuged for 10 minutes at 16,000×g. The supernatants were now transferred into new sample tubes. The extraction of the protein pellets was repeated by the addition of another 100 μL methanol and centrifugation (1,400 rpm) for 5 min. After centrifugation at 16,000×g for 10 min, both methanolic fractions were brought together and evaporated under reduced pressure to dryness. The dried residues were reconstituted in 50 μL water with a content of 0.0075% acetic acid that was ultra-sonicated for 10 min, followed by 5 min centrifugation (1,400 rpm) and centrifugation for 5 min at 16,000×g. The LC-MS/MS analyses of the supernatants were performed with an Agilent 1260 Infinity LC-System in connection with an Agilent 6490 Triple Quadrupole Mass Spectrometer (both from Waldbronn, Germany) that was connected to an electrospray ion source in the positive ion mode (ESI+). Chromatographic conditions and settings of the ESI source were as described for the quantification of dC and 5-mdC [62]. As a separation column, an Agilent Poroshell 120 EC-C18 (2.7 μm, 3.0×150 mm) was used, the injection volume was 5 μL. The quantification of 5-hmdC with respect to the stable isotope-marked standard that were both eluted at 4.9 min from the LC column (the retention times of dC and 5-mdC were 4.7 or 6.0 min, respectively), was performed with the aid of the Multiple Reaction Monitoring (MRM) approach. The following mass transitions (loss of 2′-desoxyribose) as quantifiers (optimized collision energies in parentheses) were used: 5-HmdC: m/z 258.1>142.0 (8 eV) and 5-HmdC-d3: m/z 261.1>145.0 (8 eV). For clear identification, further mass transitions were recorded. The retention time for every one of the four analyzed mass transitions was 50 ms.

### Tests of the Human IDH1 (R132H/+) HCT116 Cell Line

FIG. 29 shows the results of an MTT cytotoxicity assay that was performed as follows:
Incubation with DKA in increasing concentrations (100 μM-10 mM) for 24 h, 48 h, and 72 h
Colon carcinoma cell line HCT116 with heterozygotous mutation IDH1-(R132H/+)-MT and HCT116-IDH1-WT

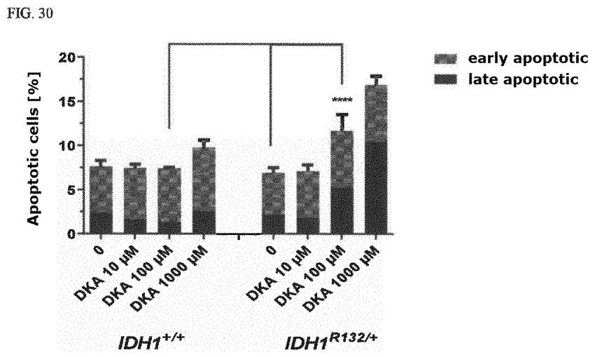

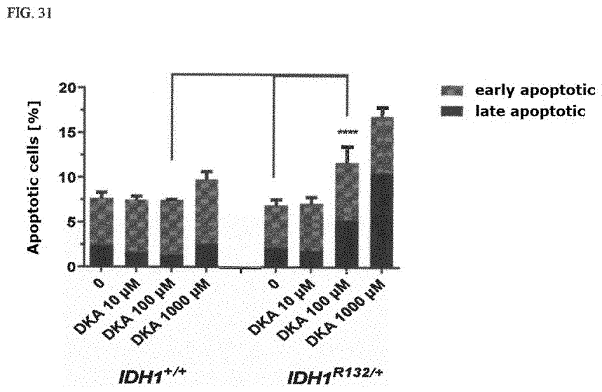

FIG. 30 shows the results of an apoptosis assay that was performed as follows:
72 h incubation with DKA in increasing concentrations (100 μM-10 mM)
Colon carcinoma cell line HCT116 with heterozygotous mutation IDH1-(R132H/+)-MT and HCT116-IDH1-WT
   Statistics: Two-way ANOVA with Dunnett's correction-****=p<0.0001

FIG. 31 shows the results of another apoptosis assay that was performed as follows:
48 h incubation DKA with increasing concentrations (100 μM-10 mM)+24 h TNFa (10 ng/mL)/CHX (1 μG/mL)
Colon carcinoma cell line HCT116 with heterozygotous mutation IDH1-(R132H/+)-MT and HCT116-IDH1-WT
   Statistics: Two-way ANOVA with Dunnett's correction-****=p<0.0001; ***=p<0.001

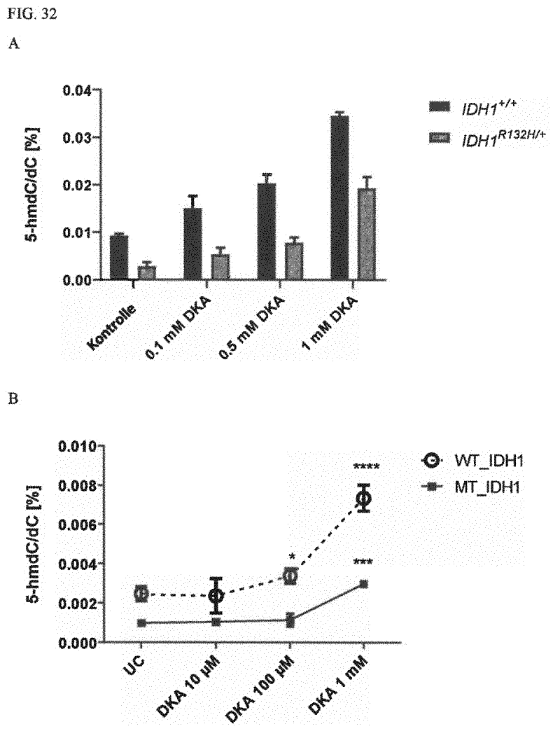

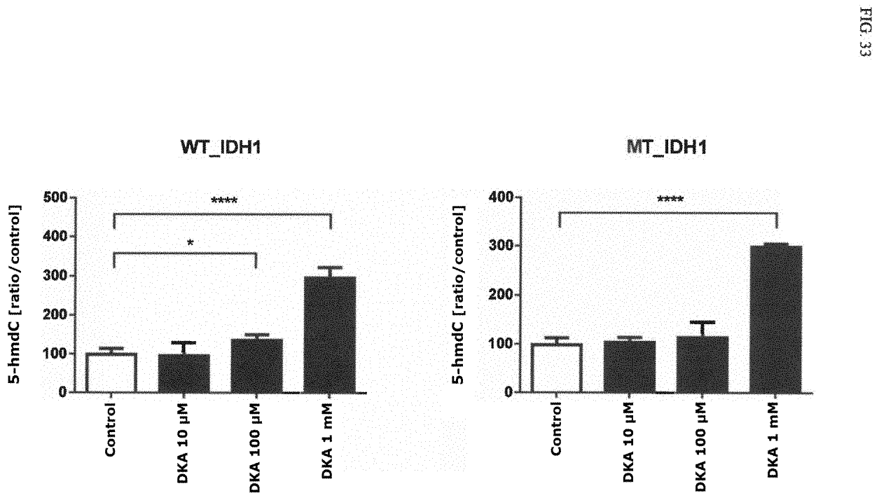

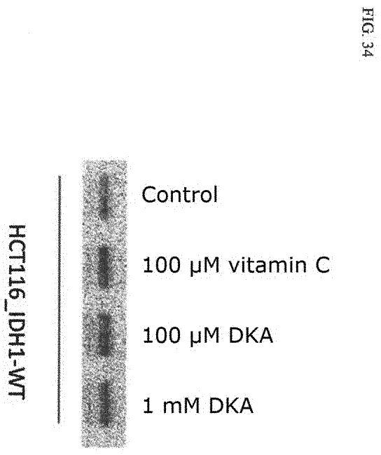

FIG. 32 shows the results of hmdC measurements (different representations) that were performed as follows:
72 h incubation with DKA in increasing concentrations (100 μM-10 mM)
Colon carcinoma cell line HCT116 with heterozygotous mutation IDH1-(R132H/+)-MT and HCT116-IDH1-WT
   Statistics: Two-way ANOVA with Dunnett's correction-****=p<0.0001; *=p<0.05

FIG. 33 shows the results of hmdC measurements (different representations) that were performed as follows:

hmdC measurement (other representation—in % relative to the untreated control as 100%)
72 h incubation with DKA in increasing concentrations (10 μM-1 mM)
Colon carcinoma cell line HCT116 with heterozygotous mutation IDH1-(R132H/+)-MT and HCT116-IDH1-WT
   Statistics: Two-way ANOVA with Dunnett's correction-****=p<0.0001; *=p<0.05

FIG. 34 shows a western blot with HCT116 cells that was performed as follows:
72 h incubation with DKA in increasing concentrations (100 μM-1 mM)
Colon carcinoma cell line HCT116 with heterozygotous mutation IDH1-(R132H/+)-MT and HCT116-IDH1-WT

With the examples, it could be shown that the claim of a co-substrate replacement and modulation with an atoxic exemplary substance such as DKG is successful. The effects as well as secondary apoptosis indicate a reconstruction of the cell metabolism.

Due to the increased or modified requirement of energy of tumor cells, these are not capable anymore to generate this from normal metabolisms and by the treatment, come therefore into a secondary apoptosis. The generation of activity of the TETs leads to demethylation of the cytosines and reversal of epigenetic changes, connected with the regeneration of tumor suppressor genes.

All this shows the functionality of co-substrate replacement (modulators) for affecting DIOs and the different pathological conditions associated therewith.

Human DIOs, in particular those which regulate the transcription, are subject matter of current research approaches for therapeutic points of attack for different anemias and cancer diseases [63-66]. The DIOs affect and regulate numerous proteins, as can be derived from a multitude of reactions indicated above [67].

Known and putative 2-OG-dependent dioxygenases in the GenBank DNA database are listed in Table 4. According to the present invention, these come into consideration for the modulation:

TABLE 4

| DNA/RNA | JmjC domain | JmjC domain | Proline/lysine | Other |
|---|---|---|---|---|
| modification | including | including | hydrolases | hydrolases |
| TET1 | KDM2A | KDM7A | ELGN1 | ASPH |
| TET2 | KDM2B | KDM8 | ELGN2 | ASPHD1 |
| TET3 | KDM3A | HR | ELGN3 | ASPHD2 |
| ABH1 | KDM3B | JARID2 | P4HA1 | BBOX1 |
| ABH2 | KDM4A | JHDM1C | P4HA2 | FIH1 |
| ABH2 | KDM4B | JMJD1C | P4HA3 | HSPBAP1 |
| ABH4 | KDM4C | JMJD4 | P4HB | OGFOD1 |
| ABH5 | KDM4D | JMJD6 | P4HTM | OGFOD2 |
| ABH6 | KDM5A | JMJD7 | PLOD1 | PAHX- |
| FTO | KDM5B | JMJD8 | PLOD2 | PHYH |
| | KDM5C | MINA | PLOD3 | PHYHD1 |
| | KDM5D | NO66 | LEPRE1 | |
| | KDM6A | PHF2 | LEPREL1 | |
| | KDM6B | PHF8 | LEPREL2 | |
| | | UTY | BBOX2 | |

### REFERENCES

1. Flashman, E. and C. J. Schofield, The most versatile of all reactive intermediates? Nature Chemical Biology, 2007. 3: p. 86.
2. Hausinger, R. P., Fe(11)/α-Ketoglutarate-Dependent Hydroxylases and Related Enzymes. Critical Reviews in Biochemistry and Molecular Biology, 2004. 39(1): p. 21-68.
3. Perry, C., et al., Rieske non-heme iron-dependent oxygenases catalyse diverse reactions in natural product biosynthesis. Natural Product Reports, 2018. 35(7): p. 622-632.
4. Loenarz, C. and C. J. Schofield, Physiological and biochemical aspects of hydroxylations and demethylations catalyzed by human 2-oxoglutarate oxygenases. Trends in Biochemical Sciences, 2011. 36(1): p. 7-18.
5. Prescott, A. G. and M. D. Lloyd, The iron(ll) and 2-oxoacid-dependent dioxygenases and their role in metabolism. Natural Product Reports, 2000. 17(4): p. 367-383.
6. Scotti, J. S., et al., Human oxygen sensing may have origins in prokaryotic elongation factor Tu prolyl-hydroxylation. Proceedings of the National Academy of Sciences, 2014. 111(37): p. 13331-13336.
7. Clifton, I. J., et al., Crystal Structure of Carbapenem Synthase (CarC). Journal of Biological Chemistry, 2003. 278(23): p. 20843-20850.
8. Farrow, S. C. and P. J. Facchini, Functional diversity of 2-oxoglutarate/Fe(ll)-dependent dioxygenases in plant metabolism. Frontiers in Plant Science, 2014. 5: p. 524.
9. Cheng, A. X., et al., The function and catalysis of 2-oxoglutarate-dependent oxygenases involved in plant flavonoid biosynthesis. Int J Mol Sei, 2014. 15(1): p. 1080-95.
10. Zhang, Z., et al., Crystal Structure and Mechanistic Implications of 1-Aminocyclopropane-1-Carboxylic Acid Oxidase—The Ethylene-Forming Enzyme. Chemistry & Biology, 2004. 11(10): p. 1383-1394.
11. Myllyharju, J., Prolyl 4-hydroxyiases, the key enzymes of Collagen biosynthesis. Matrix Biology, 2003. 22(1): p. 15-24.
12. Leung, I. K. H., et al., Structural and Mechanistic Studies on y-Butyrobetaine Hydroxylase. Chemistry & Biology, 2010. 17(12): p. 1316-1324.
13. Markolovic, S., S. E. Wilkins, and C. J. Schofield, Protein Hydroxylation Catalyzed by 2-Oxoglutarate-dependent Oxygenases. Journal of Biological Chemistry, 2015. 290(34): p. 20712-20722.
14. Walport, L. J., R. J. Hopkinson, and C. J. Schofield, Mechanisms of human histone and nucleic acid demethylases. Current Opinion in Chemical Biology, 2012. 16(5): p. 525-534.
15. Salminen, A., A. Kauppinen, and K. Kaarniranta, 2-Oxoglutarate-dependent dioxygenases are sensors of energy metabolism, oxygen availability, and iron homeostasis: potential role in the regulation of aging process. Cellular and Molecular Life Sciences, 2015. 72(20): p. 3897-3914.
16. Martinez, S. and R. P. Hausinger, Catalytic Mechanisms of Fe(ll)- and 2-Oxoglutarate-dependent Oxygenases. Journal of Biological Chemistry, 2015. 290(34): p. 20702-20711.
17. Myllylä, R., et al., Ascorbate is consumed stoichiometrically in the uncoupled reactions catalyzed by prolyl 4-hydroxylase and lysyl hydroxylase. Journal of Biological Chemistry, 1984. 259(9): p. 5403-5405.
18. Flashman, E., et al., Investigating the dependence of the hypoxia-inducible factor hydroxylases (factor inhibiting HIF and prolyl hydroxylase domain 2) on ascorbate and other reducing agents. Biochem J, 2010. 427(1): p. 135-42.
19. Lappin, T. and N. Masson, Two antioxidants are better than one. Blood, 2011. 117(20): p. 5276.
20. Mastrangelo, D., et al., Mechanisms of anti-cancer effects of ascorbate: Cytotoxic activity and epigenetic modulation. Blood Cells, Molecules, and Diseases, 2018. 69: p. 57-64.
21. Shenoy, N., et al., Ascorbic Acid in Cancer Treatment: Let the Phoenix Fly. Cancer Cell, 2018.
22. Solomon, E. I., A. Decker, and N. Lehnert, Non-heme iron enzymes: Contrasts to heme catalysis. Proceedings of the National Academy of Sciences, 2003. 100(7): p. 3589.
23. Huang, C. W., et al., The different catalytic roles of the metal-binding ligands in human 4-hydroxyphenylpyruvate dioxygenase. Biochem J, 2016. 473(9): p. 1179-89.
24. Hewitson, K. S., et al., Oxidation by 2-oxoglutarate oxygenases: non-haem iron Systems in catalysis and signalling. Philosophical Transactions of the Royal Society A: Mathematical, Physical and Engineering Sciences, 2005. 363(1829): p. 807.
25. Raili, M., T. Leena, and K. K. I., Mechanism of the Prolyl Hydroxylase Reaction. European Journal of Biochemistry, 1977. 80(2): p. 349-357.
26. Price, J. C., et al., The First Direct Characterization of a High-Valent Iron Intermediate in the Reaction of an α-Ketoglutarate-Dependent Dioxygenase: A High-Spin Fe(IV) Complex in Taurine/α-Ketoglutarate Dioxygenase (TauD) from Escherichia coli. Biochemistry, 2003. 42(24): p. 7497-7508.
27. Proshlyakov, D. A., et al., Direct Detection of Oxygen Intermediates in the Non-Heme Fe Enzyme Taurine/α-Ketoglutarate Dioxygenase. Journal of the American Chemical Society, 2004. 126(4): p. 1022-1023.
28. Welford, R. W., et al., Incorporation of oxygen into the succinate co-product of iron(ll) and 2-oxoglutarate dependent oxygenases from bacteria, plants and humans. FEBS Lett, 2005. 579(23): p. 5170-4.
29. Grzyska, P. K., et al., Insight into the mechanism of an iron dioxygenase by resolution of Steps following the FelV=HO species. Proc Natl Acad Sci USA, 2010. 107(9): p. 3982-7.
30. Valegärd, K., et al., The structural basis of Cephalosporin formation in a mononuclear ferrous enzyme. Nature Structural & Molecular Biology, 2003. 11: p. 95.
31. Wiek, C. R., et al., Structural Insight into the Prolyl Hydroxylase PHD2: A Molecular Dynamics and DFT Study. European Journal of Inorganic Chemistry, 2012. 2012(31): p. 4973-4985.
32. Tarhonskaya, H., et al., Studies on Deacetoxycephalosporin C Synthase Support a Consensus Mechanism for 2-Oxoglutarate Dependent Oxygenases. Biochemistry, 2014. 53(15): p. 2483-2493.
33. McDonough, M. A., et al., Structural studies on human 2-oxoglutarate dependent oxygenases. Current Opinion in Structural Biology, 2010. 20(6): p. 659-672.
34. Clifton, I. J., et al., Structural studies on 2-oxoglutarate oxygenases and related double-stranded beta-helix fold proteins. J Inorg Biochem, 2006. 100(4): p. 644-69.
35. L, H. E. and J. L. Que, The 2-His-l-Carboxylate Facial Triad—An Emerging Structural Motif in Mononuclear Non-Heme Iron(ll) Enzymes. European Journal of Biochemistry, 1997. 250(3): p. 625-629.
36. Chowdhury, R., et al., Structural basis for binding of hypoxia-inducible factor to the oxygen-sensing prolyl hydroxylases. Structure, 2009. 17(7): p. 981-9.
37. Deng, G., et al., Novel complex crystal structure of prolyl hydroxylase domain-containing protein 2 (PHD2): 2,8-Diazaspiro[4.5]decan-l-ones as potent, orally bioavailable PHD2 Inhibitors. Bioorg Med Chem, 2013. 21(21): p. 6349-58.
38. Chowdhury, R., et al., Structural basis for oxygen degradation domain selectivity of the HIF prolyl hydroxylases. Nature Communications, 2016. 7: p. 12673.
39. Willam, C., et al., The prolyl hydroxylase enzymes that act as oxygen sensors regulating destruction of hypoxia-inducible factor alpha. Adv Enzyme Regul, 2004. 44: p. 75-92.
40. Rabe, P., et al., Roles of 2-oxoglutarate oxygenases and isopenicillin N synthase in 6-lactam biosynthesis. Natural Product Reports, 2018.
41. Hegg, E. L. and L. Q. Jr, The 2-His-l-Carboxylate Facial Triad—An Emerging Structural Motif in Mononuclear Non-Heme Iron(ll) Enzymes. European Journal of Biochemistry, 1997. 250(3): p. 625-629.
42. Kal, S. and L. Que, Dioxygen activation by nonheme iron enzymes with the 2-His-1-carboxylate facial triad that generate high-valent oxoiron oxidants. JBIC Journal of Biological Inorganic Chemistry, 2017. 22(2): p. 339-365.
43. Xu, W., et al., Oncometabolite 2-Hydroxyglutarate Is a Competitive Inhibitor of α-Ketoglutarate-Dependent Dioxygenases. Cancer Cell, 2011. 19(1): p. 17-30.
44. Xu, W., et al., Oncometabolite 2-hydroxygiutarate is a competitive inhibitor of alpha-Ketoglutarate-dependent dioxygenases. Cancer Cell, 2011. 19(1): p. 17-30.
45. Elkashef, S. M., et al., IDH Mutation, Competitive Inhibition of FTO, and RNA Methylation. Cancer Cell, 2017. 31(5): p. 619-620.
46. Flavahan, W. A., et al., Insulator dysfunction and oncogene activation in IDH mutant gliomas. Nature, 2016. 529(7584): p. 110-4.
47. Buege, M. J., A. J. DiPippo, and C. D. DiNardo, Evolving Treatment Strategies for Elderly Leukemia Patients with IDH Mutations. Cancers (Basel), 2018. 10(6).
48. Rautio, J., et al., The expanding role of prodrugs in Contemporary drug design and development. Nat Rev Drug Discov, 2018. 17(8): p. 559-587.
49. van Gorkom, G., et al., Influence of Vitamin C on Lymphocytes: An OverView. Antioxidants, 2018. 7(3): p. 41.
50. Cimmino, L, B. G. Neel, and I. Aifantis, Vitamin C in Stern Cell Reprogramming and Cancer. Trends Cell Biol, 2018.
51. Li, X., et al., Ten-eleven translocation 2 demethylates the MMP9 promoter, and its down-regulation in preeclampsia impairs trophoblast migration and invasion. J Biol Chem, 2018.
52. Sant, D. W., et al., Vitamin C promotes apoptosis in breast cancer cells by increasing TRAIL expression. Scientific Reports, 2018. 8(1): p. 5306.
53. Park, S., et al., Vitamin C in Cancer: A Metabolomics Perspective. Frontiers in Physiology, 2018. 9(762).
54. Monacelli, F., et al., Vitamin C, Aging and Alzheimer's Disease. Nutrients, 2017. 9(7).
55. Ebata, K. T., et al., Vitamin C induces specific demethylation of H3K9me2 in mouse embryonic stem cells via Kdm3a/b. Epigenetics & Chromatin, 2017. 10(1): p. 36.
56. D'Aniello, C., et al., Vitamin C and I-Proline Antagonistic Effects Capture Alternative States in the Pluripotency Continuum. Stem Cell Reports, 2017. 8(1): p. 1-10.
57. Schonberger, K. and N. Cabezas-Wallscheid, Vitamin C: Cing a New Way to Fight Leukemia. Cell Stem Cell, 2017. 21(5): p. 561-563.
58. Unlu, A., et al., High-dose vitamin C and cancer. Journal of Oncological Science, 2016. 1: p. 10-12.
59. Li, R., Vitamin C, a Multi-Tasking Molecule, Finds a Molecular Target in Killing Cancer Cells. React Oxyg Species (Apex), 2016. 1(2): p. 141-156.
60. Levine, M. and P.-C. Violet, Data Triumph at C. Cancer Cell, 2017. 31(4): p. 467-469.
61. Monfort, A. and A. Wutz, Breathing-in epigenetic change with vitamin C. EMBO Rep, 2013. 14(4): p. 337-46.
62. Schumacher, F., et al., Optimized enzymatic hydrolysis of DNA for LC-MS/MS analyses of adducts of 1-methoxy-3-indolylmethyl glucosinolate and methyleugenol. Anal Biochem, 2013. 434(1): p. 4-11.
63. Islam, M. S., et al., 2-Oxoglutarate-Dependent Oxygenases. Annual Review of Biochemistry, 2018. 87(1): p. 585-620.
64. Lv, X., et al., The role of hypoxia-inducible factors in tumor angiogenesis and cell metabolism. Genes & Diseases, 2017. 4(1): p. 19-24.
65. Wigerup, C., S. Pahlman, and D. Bexell, Therapeutic targeting of hypoxia and hypoxia-inducible factors in cancer. Pharmacol Ther, 2016. 164: p. 152-69.
66. Xia, Y., H. K. Choi, and K. Lee, Recent advances in hypoxia-inducible factor (HIF)-1 inhibitors. Eur J Med Chem, 2012. 49: p. 24-40.
67. Semenza, G. L., A compendium of proteins that interact with HIF-lalpha. Exp Cell Res, 2017. 356(2): p. 128-135.

1. A compound selected from the compounds according to Formula (I) or Formula (II) 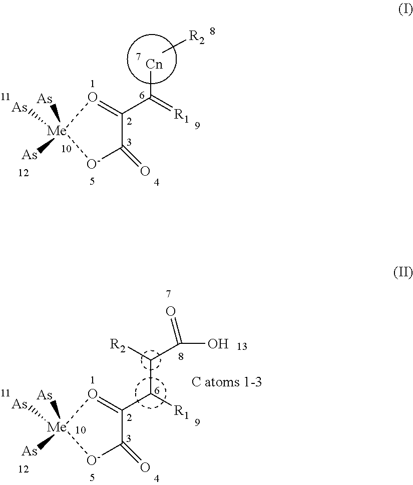
wherein
As represent the amino acids from the binding pocket of the enzyme;
Me is a metal from the catalytic center;
R₁ and R₂ are oxygen (hydroxyl) or carboxyl groups, halogens, in particular fluorine, chlorine, or iodine, a mono- or poly-halogenated methyl group, in particular, CH₂F up to CF₃; and
Cn represents a C atom, a heteroatom, or the bridge to a heterocycle.

2. The compound of claim 1, wherein R₁ is hydrogen or a CH₂R₃ group, wherein R₃ is hydrogen or oxygen (hydroxyl, carbonyl) or a shorter C-chain (C₁ to C₄).

3. The compound of claim 1, wherein the compound is part of a ring system.

4. The compound of claim 3, wherein the ring size is between 3 and 5 atoms with at least one heteroatom.

5. The compound of claim 1, wherein C₇ consists of a carbon chain with up to 5 atoms and contains double bonds.

6. The compound of claim 1, wherein R₂ represents a carboxylic acid.

7. The compound of claim 1, wherein in a compound according to Formula (II), R₂ represents a hydrogen atom, a methyl group, an alkyl group with up to 6 C atoms that may be branched saturated or unsaturated or may themselves also contain a heteroatom.

8. The compound of claim 1, wherein in a compound according to Formula (II), between R₁ and R₂, a bridge-forming cyclic structure is arranged that contains unsaturated or saturated heteroatoms.

9. The compound of one of claim 1, wherein mixtures of the compounds according to Formula (I) and Formula (II) are used.

10. The compound of one of claim 1, wherein pharmaceutically acceptable salts and tautomers of the respective compound are used alone or in combination in predefined mixing ratios.

11. A use of a compound of claim 1 as a drug.

12. A use of a compound of claim 1 as a co-substrate with other active ingredients in a drug.

13. The use of claim 12, wherein the other active ingredients are selected from the group comprising chemotherapeutics, cytostatics, such as alkylants, antimetabolites, topoisomerase inhibitors, mitose inhibitors, antibiotics, antibodies, kinase inhibitors, proteasome inhibitors, and supportive medicinal substances of the tumor therapy such as, in particular, interferons, cytokines, tumor necrosis factor, and IDH inhibitors.

14. The use of a compound of claim 1 as a drug for the prevention, treatment, or follow-up of cancer diseases, of neurodegenerative diseases and of congenital or acquired metabolic disorders.

15. The use of a compound of claim 1 for the preparation of a drug for the prevention, treatment, or follow-up of cancer diseases, of neurodegenerative diseases and of congenital or acquired metabolic disorders.

16. A drug comprising a compound of claim 1.


SEQUENCE LISTING (20 sequences)

SEQ ID NO: 1 - DNA, 20 bp, Artificial Sequence
  Forward Primer hHMBS
  misc_feature: Forward Primer hHMBS
accaaggagcttgaacatgc

SEQ ID NO: 2 - DNA, 21 bp, Artificial Sequence
  Reverse Primer hHMBS
  misc_feature: Reverse Primer hHMBS
gaaagacaacagcatcatgag

SEQ ID NO: 3 - DNA, 20 bp, Artificial Sequence
  Forward Primer hDNMT1
  misc_feature: Forward Primer hDNMT1
acctggctaaagtcaaatcc

SEQ ID NO: 4 - DNA, 20 bp, Artificial Sequence
  Reverse primer hDNMT1
  misc_feature: Reverse primer hDNMT1
attcacttcccggttgtaag

SEQ ID NO: 5 - DNA, 20 bp, Artificial Sequence
  Forward Primer hDNMT3a
  misc_feature: Forward Primer hDNMT3a
actacatcagcaagcgcaag

SEQ ID NO: 6 - DNA, 20 bp, Artificial Sequence
  Reverse primer hDNMT3a
  misc_feature: Reverse primer hDNMT3a
catccaccaagacacaatgc

SEQ ID NO: 7 - DNA, 20 bp, Artificial Sequence
  Forward Primer hDNMT3b
  misc_feature: Forward Primer hDNMT3b
ccagctcttaccttaccatc

SEQ ID NO: 8 - DNA, 20 bp, Artificial Sequence
  Reverse Primer hDNMT3b
  misc_feature: Reverse Primer hDNMT3b
cagacatagcctgtcgcttg

SEQ ID NO: 9 - DNA, 20 bp, Artificial Sequence
  Forward Primer hTET1
  misc_feature: Reverse Primer hTET1
gctgctgtcagggaaatcat

SEQ ID NO: 10 - DNA, 20 bp, Artificial Sequence
  Reverse Primer hTET1
  misc_feature: Reverse Primer hTET1
accatcacagcagttggaca

SEQ ID NO: 11 - DNA, 20 bp, Artificial Sequence
  Forward Primer hTET2
  misc_feature: Forward Primer hTET2
ccaataggacatgatccagg

SEQ ID NO: 12 - DNA, 20 bp, Artificial Sequence
  Reverse Primer hTET2
  misc_feature: Reverse Primer hTET2
tctggatgagctctctcagg

SEQ ID NO: 13 - DNA, 20 bp, Artificial Sequence
  Forward Primer hTET3
  misc_feature: Forward Primer hTET3
tcggagacaccctctaccag

SEQ ID NO: 14 - DNA, 20 bp, Artificial Sequence
  Reverse Primer hTET3
  misc_feature: Reverse Primer hTET3
cttgcagccgttgaagtaca

SEQ ID NO: 15 - DNA, 18 bp, Artificial Sequence
  Forward primer CDKN2A (p16)
  misc_feature: Forward primer CDKN2A (p16)
gagcagcatggagccttc

SEQ ID NO: 16 - DNA, 20 bp, Artificial Sequence
  Reverse Primer CDKN2A (p16)
  misc_feature: Reverse Primer CDKN2A (p16)
cctccgaccgtaactattcg

SEQ ID NO: 17 - DNA, 20 bp, Artificial Sequence
  Forward primer CDKN1A (p21)
  misc_feature: Forward primer CDKN1A (p21)
agtggacagcgagcagctga

SEQ ID NO: 18 - DNA, 23 bp, Artificial Sequence
  Reverse primer CDKN1A (p21)
  misc_feature: Reverse primer CDKN1A (p21)
tagaaatctgtcatgctggtctg

SEQ ID NO: 19 - DNA, 23 bp, Artificial Sequence
  Forward Primer CDKN1B (p27)
  misc_feature: Forward Primer CDKN1B (p27)
aaacgtgcgagtgtctaacggga

SEQ ID NO: 20 - DNA, 23 bp, Artificial Sequence
  Reverse Primer CDKN1B (p27)
  misc_feature: Reverse Primer CDKN1B (p27)
cgcttccttattcctgcgcattg